(19) United States
(12) Patent Application Publication
(10) Pub. No.: US 2021/0145983 A1
(43) Pub. Date: May 20, 2021

(54) METHODS AND COMPOSITIONS OF INHIBITING CSF1R FOR TREATING ALLERGIC INFLAMMATION

(71) Applicant: THE BOARD OF TRUSTEES OF THE UNIVERSITY OF ILLINOIS, URBANA, IL (US)

(72) Inventors: Gye Young Park, Chicago, IL (US); Hyung-Geun Moon, Chicago, IL (US)

(21) Appl. No.: 17/047,929

(22) PCT Filed: Apr. 18, 2019
(86) PCT No.: PCT/US2019/028123

§ 371 Date: Oct. 15, 2020

Related U.S. Application Data
(60) Provisional application No. 62/750,352, filed on Oct. 25, 2018.
(60) Provisional application No. 62/659,791, filed on Apr. 19, 2018.

(51) Int. Cl. A61K 47/69; A61P 11/06; A61K 31/505; A61K 31/444; A61K 9/00; A61K 9/06; A61K 47/59

(52) U.S. Cl. CPC A61K 47/6951; A61P 11/06; A61K 31/505; A61K 31/444; A61K 9/0043; A61K 47/595; A61K 9/0048; A61K 9/0014; A61K 9/06; A61K 47/6927; A61K 9/0073

(57) ABSTRACT

In one aspect, the disclosure relates to methods of treating a disease or condition characterized by allergic inflammation, including, but not limited to, asthma, allergic conjunctivitis, allergic dermatitis, allergic esophagitis, allergic rhinitis, allergen-specific serum IgE production, allergic lung and airway inflammation and airway hyper-responsiveness (AHR) with minimal pulmonary adverse reaction, by administration of at least one CSF1R inhibitor and optionally a further therapeutic agent that is an anti-inflammatory drug and/or respiratory drug. In some aspects, the disclosed CSF1R inhibitors useful in the treatment of characterized by allergic inflammation can be administered using a disclosed pharmaceutical composition. This abstract is intended as a scanning tool for purposes of searching in the particular art and is not intended to be limiting of the present disclosure.

### CROSS-REFERENCE TO RELATED APPLICATIONS

This application claims the benefit of U.S. Provisional Application No. 62/659,791, filed on Apr. 19, 2018, and U.S. Provisional Application No. 62/750,352, filed Oct. 25, 2018, which are incorporated herein by reference in their entireties.

### STATEMENT REGARDING FEDERALLY SPONSORED RESEARCH

This invention was made with Government support under grant number HL126852 awarded by the National Institute of Health. The government has certain rights in the invention.

### BACKGROUND

Despite current standard medical treatment, a large number of patients with asthma remain symptomatic with long-term disabilities. There is a desperate need for developing a new treatment strategy for asthma. The events at the mucosal layer of airways are crucial to driving the development of Th2 immune responses. The mechanisms by which Th2 immune-mediated allergic inflammation is initiated at the airway mucosa are better understood (Lambrecht B N. et al. Immunity 2009 31:412-424; Hammad H, et al. Immunity 2015 43:29-40). A therapeutic strategy targeting the early initial events of the mucosal immune reaction against aeroallergens could have greater therapeutic benefits because it could abolish all of the subsequent IgE- and Th2-mediated aspects of allergic inflammation in the lung.

Asthma is a chronic airway disease with intermittent flare-ups. People become asthmatic through the process of ‘sensitization’ against specific allergen(s) such as house dust mite and pollens. Once sensitized, individuals will develop airway inflammation and shortness of breath with re-exposure to the sensitizing allergen. Therapies targeting allergen sensitization have shown efficacy in controlling asthma (Busse W W. et al. N Engl J Med 2011 364:1005-1015: Teach S J. et al. J Allergy Clin Immunol 2015 136:1476-1485). The sensitization process for aeroallergens is mainly carried out by two major cell types, airway epithelial cells and dendritic cells.

Airway epithelial cells (AECs) are a gateway for sensing aeroallergens through first direct contact with inhaled particles (Lambrecht B N, et al. J Allergy Clin Immunol 2014 134:499-507). Pattern recognition receptors including toll like receptors of AECs are required to recognize allergens and produce subsequent allergic inflammatory signals. Upon being activated by aeroallergens, AECs produce cytokines, chemokines and endogenous danger signals toward the basal side as well as luminal side and alter the micro-environmental milieu to activate innate immune cells. AECs secrete innate cytokines which have a critical role in recruiting immune cells and skewing the immune reaction towards a predominant Th2 pattern (Hammad H. et al. Immunity 2015 43:29-40). On the luminal side, it has been shown that AECs secreted chemokines into the alveolar space, recruiting monocyte-derived alveolar macrophages upon allergen challenge (Lee Y G. et al. Am J Respir Cell Mol Biol 2015 52:772-784; Zaslona Z, et al. J Immunol 2014 193:4245-4253). These findings indicate that AECs regulate the micro-environmental milieu of the airway in favor of allergic inflammation in response to aeroallergens.

During the sensitization, dendritic cells (DCs) take up and process the “invading” allergen and migrate to the regional lymph nodes (LNs), resulting in the establishment of allergen-specific Th2 memory. DCs form an essential interface between this innate and adaptive immunity, and play a key role in primary sensitization and the production of antigen-specific IgE (Lambrecht B N, et al. Immunity 2009 31:412-424). DCs traffic inhaled allergens to the LNs where they launch an antigen-specific adaptive immune response involving T and B cells (Joffre O, et al. Immunol Rev 2009 227:234-247). Conventional DCs (cDCs) play a key role in antigen presentation in various types of inflammation, cDCs express higher CCR7 which is involved in activation and homing of DCs to lymph nodes. In both human and mouse, two main distinct subsets of cDC, cDC1 and cDC2, possess unique characteristics and properties, cDC2 strongly depend on IRF4 and express multiple pattern recognition receptors, whereas cDC1 depend on IRF8 and activate CD8+ T cells. Especially, cDC2 have a critical role in MHC class 2-dependent antigen presentation and are more effective carriers of allergen to the regional LNs in animal models of asthma (Plantinga M, et al. Immunity 2013 38:322-335).

However, it has not been fully elucidated how AECs and DCs interact in the process of sensing aeroallergens. AECs and DCs cooperate to facilitate allergen sensitization (Moon H-G, et al. Immunity 2018 49:275-287.e275). AECs secrete CSF1 into the alveolar space in response to aeroallergen. CSF1 was markedly elevated in the BAL fluids of patients with asthma after being challenged with a sensitizing allergen via bronchoscopy. With the relevant animal models, it was determined that AECs are the primary source of CSF1 in BAL fluid. The epithelial-secreted CSF1 activated DCs, particularly cDC2, and facilitated mobilization to regional LNs by regulating the DC expression of CCR7, a homing chemokine receptor to LNs (Moon H-G, et al. Immunity 2018 49:275-287.e275). This is the earliest event in the mucosal immune response to aeroallergen, but also occurs as a memory response that sustains asthmatic airway disease. Blocking this process could abolish sensitization to allergens and subsequent allergic inflammation upon repeated allergen exposure.

Despite advances in inflammatory research, e.g., allergic inflammation, there is still a scarcity of compounds that are both potent, efficacious, and selective therapeutic agents useful in the treatment such disorders. These needs and other needs are satisfied by the present disclosure.

### SUMMARY

In one aspect, the disclosure relates to methods of treating a disease or condition characterized by allergic inflammation, including, but not limited to, asthma, allergic conjunctivitis, allergic dermatitis, allergic esophagitis, allergic rhinitis, allergen-specific serum IgE production, allergic lung and airway inflammation and airway hyper-responsiveness (AHR) with minimal pulmonary adverse reaction, by administration of at least one CSF1R inhibitor and optionally a further therapeutic agent that is an anti-inflammatory drug and/or respiratory drug. In some aspects, the disclosed CSF1R inhibitors useful in the treatment of characterized by allergic inflammation can be administered using a disclosed pharmaceutical composition.

As disclosed herein, the CSF1-CSF1R pathway is critical for establishing and maintaining a Th2 memory response in regional lymph nodes. Intranasal delivery of a nanoparticle carrying a CSF1R antagonist has efficacy and shows favorable pharmacokinetics without significant adverse effects. Therefore, CSF1R inhibition is a promising new therapeutic approach for allergic inflammation, particularly allergic asthma.

Accordingly, in an aspect, this disclosure is a method of treating a disease or condition characterized by allergic inflammation in a subject comprising administering to the subject an effective amount of a small molecule CSF1R inhibitor.

In another aspect, this disclosure is a method of modulating allergic inflammation in a subject exposed to sensitized allergen comprising administering to the subject an effective amount of a small molecule CSF1R inhibitor.

In certain aspects, the CSF1R inhibitor is selected from PLX3397, DCC-3014, BLZ945, GW2580, PLX647 and ARRY-382, or a pharmaceutically acceptable salt thereof. In certain particular aspects, the CSF1R inhibitor is GW2580 or PLX3397, or a pharmaceutically acceptable salt thereof.

In other aspects, a microparticle comprising the small molecule CSF1R inhibitor is administered to the subject. In other aspects, the microparticle further comprises β-cyclodextrin conjugated epsilon-polylysine.

In an aspect, the disease or condition characterized by allergic inflammation is selected from asthma, allergic conjunctivitis, allergic dermatitis, allergic esophagitis and allergic rhinitis. In another aspect, the CSF1R inhibitor is administered to the patient during an acute allergic flare-up. In another aspect, the CSF1R inhibitor is administered to the patient prior to an acute allergic flare-up.

In other aspects, the disease or condition characterized by allergic inflammation is selected from asthma, allergic rhinitis, inflammation is allergic rhinitis, allergic conjunctivitis, allergic dermatitis and allergic esophagitis.

In other aspects, the asthma or allergic rhinitis is treated by administering to the subject an aerosol formulation comprising the CSF1R inhibitor.

In other aspects, the allergic conjunctivitis is treated by administering to the subject an ophthalmic composition comprising the CSF1R inhibitor.

In other aspects, the allergic dermatitis is treated by administering to the subject a topical cream or ointment comprising the CSF1R inhibitor.

In other aspects, the allergic esophagitis is treated by administering to the subject an oral formulation comprising the CSF1R inhibitor.

In another aspect, this disclosure is a microparticle comprising the CSF1R inhibitor or a pharmaceutically acceptable salt thereof. In an aspect, the microparticle further comprises β-cyclodextrin conjugated epsilon-poylysine.

In another aspect, this disclosure is an aerosol formulation comprising the CSF1R inhibitor or a pharmaceutically acceptable salt thereof.

In another aspect, this disclosure is an ophthalmic formulation comprising the CSF1R inhibitor or a pharmaceutically acceptable salt thereof.

In another aspect, this disclosure is a formulation for topical administration comprising a CSF1R inhibitor selected from PLX3397, DCC-3014. BLZ945, GW2580. PLX647 and ARRY-382, or a pharmaceutically acceptable salt thereof and an ophthalmic excipient.

In another aspect, this disclosure is a formulation for oral administration comprising a CSF1R inhibitor is selected from PLX3397, DCC-3014, BLZ945. GW2580, PLX647 and ARRY-382, or a pharmaceutically acceptable salt thereof.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE FIGURES

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1F shows that CSF1 and CSF1R⁺cDCs are highly enriched in the BAL fluid of the chronic DRA model. (A) Left: Experimental scheme for the chronic DRA-induced murine model of asthma (the chronic DRA model). Right: The BAL fluids were obtained at the indicated time points and the level of CSF1 in the BAL fluids was measured by ELISA. (B) By using the gating strategy as depicted in FIG. 7, cDC and CSF1R⁺cDC populations were measured in the BAL fluids over the course of the chronic DRA model. (C) The concentrations of total and DRA-reactive serum IgE were measured at the indicated time points. The DRA-reactive serum IgE is expressed as fold increase, compared to the steady state IgE level. *p<0.05, **p<0.01.

FIG. 2 shows that depletion of AEC-derived CSF1 prevents allergen sensitization and abolishes chronic allergic lung inflammation. (A) Experimental scheme for depleting AEC-derived CSF1 in the chronic DRA model using Scgb1a1-creERT; Csf1^{fi/fi} (CSF1^{ΔEC}) mice. Tamoxifen was orally administered for 5 consecutive days (one cycle) every 4 weeks. All samples were collected at the end of week 11. (B-E) Tamoxifen alone has no effect on the BAL cytokines in the absence of DRA challenge (Tm_Veh), although lowering slightly the basal BAL CSF1 level (FIG. 2B). DRA challenge induced marked increases in BAL CSF1. IL-4 and IL-13 (Veh_DRA), which were significantly reduced by Tm treatment (Tm_DRA) (2B & E). Total and DRA-reactive serum IgE concentrations were also suppressed by Tm treatment (2C). The DRA-reactive serum IgE are expressed as fold increase compared to that of the sham-treated control group (Veh_Veh). The increase in total BAL cells was suppressed by Tm treatment and the cells were mainly comprised of macrophages and lymphocytes (2D). (F) Morphometric digital pathologic analysis was performed over an entire single lung field as described in Methods. The color codes for the digital pathology indicate the following: green-inflammatory cells; yellow-area of the airway; brown-void space. The area of inflammation was markedly reduced by Tm treatment. The scale bars are 5 mm in middle and 500 μm in right panels. (G-1) Periodic acid Schiff (PAS) staining for goblet cells and immunofluorescence staining with anti-collagen and anti-α-smooth muscle actin antibodies were compared between the Tm-treated and sham-treated DRA groups. The signals were quantified by measurement of the signals per field. Scale bars represent 200 μm. *p<0.05, **p<0.01.

FIG. 3 shows that CSF1 depletion reverses established chronic allergic inflammation and airway remodeling, and suppresses Th2 memory responses in LNs. (A) Experimental scheme for depleting AEC-derived CSF1 in the well-established chronic DRA allergic inflammation model. Tm was administered for only one cycle, at week 6 of the chronic DRA model. (B) Single treatment with Tm (DRA/Tm) at week 6 reduced the BAL CSF1 concentration. (C-F) Tm treatment in the late phase of the model was able to reverse the BAL lymphocytosis, increased serum IgE, chronic lung inflammation and goblet cell metaplasia. The scale bars in left and right of (E) and (F) are 5 mm, 500 μm and 200 μm, respectively. (G) The cells from the LNs were isolated from each group, and then re-stimulated with or without DRA for 72 hours ex-vivo in culture. The cytokines in culture supernatants were measured. The re-stimulation with DRA increased the secretion of IL-4, IL-13, IL-17 and IFN-γ in the LN cultures. However, the group treated with Tm (DRA/Tm) showed a blunted response for IL-4 and IL-13, whereas IL-17 and IFN-γ levels remained unchanged. *p<0.05, **p<0.01.

FIG. 4 shows that IRF4 CSF1R′ cells are required for allergen sensitization and Th2 memory in the secondary LNs. (A) Experimental scheme for depleting CSF1R⁺IRF4⁺ antigen presenting cells in Csf1r-creERT; Irf4^{fi/fi} (Irf4^{ΔAPC}) mice. Tm2 and Tm6 indicate that the Tm administration was started at weeks 2 and 6 of the chronic DRA model as depicted. (B) Depletion of CSF1R⁺IRF4⁺ in Irf4^{ΔAPC} mice had no effect on BAL CSF1 concentration. (C-F) However, depletion of CSF1R⁺IRF4⁺ from week 2 (Tm2) resulted in a modest decrease in BAL inflammatory cells, significant reduction of total and DRA-reactive serum IgE, marked reduction of chronic lung inflammation and goblet cell metaplasia. The Tm6 group showed similar trends, but was less effective than in the Tm2 group. Left and middle scale bars in (E) are 5 mm and 500 μm, respectively. (G) Single cell suspensions of LNs were isolated from each group and re-stimulated with DRA for 72 hours. The cytokines in culture supernatants were measured. The re-stimulation with DRA boosted the secretion of IL-4, IL-13 and IL-17, compared to the sham-treated control. However, the Tm treated groups showed a blunted response for IL-4 and IL-13 production, whereas IL-17 secretion in the Tm 6 group was not affected by Tm treatment while the Tm2 group showed a mild decrease in IL-17 production. *p<0.05, **p<0.01.

FIG. 5 shows that CDPL-GW nanoparticles carrying CSF1R inhibitor block cDC2 migration and allergen sensitization. (A) The structure of CDPL-GW nanoparticle which carries 4 molecules of GW2580, a selective CSF1R inhibitor and a ZW800-1 fluorescent dye. (B-C) The experimental scheme to optimize the dose of CDPL-GW. The total AM count and annexin V⁺ AMs in BAL fluids were measured by flow cytometry. AMs and eosinophils in BAL fluids were identified by CD11c⁺SiglecF⁺, CD11c⁻SiglecF⁺ respectively. DRA challenge induced increases in BAL AMs and eosinophils, which was inhibited by the CDPL-GW treatment in a dose dependent manner. However, there was no change in the proportion of annexin V⁺ cells up to 1 ng (=1000 pg) of CDPL-GW/mouse which was a sufficient dose to suppress the BAL eosinophil recruitment and total serum IgE rise (n=4-5). (D) Migratory DCs (defined by CD45⁺lineage-CD11c⁺MHCII⁺CCR7⁺) were measured in the single cell suspensions isolated from mediastinal LNs. The number of migratory DCs in LNs gradually declined as the dose of CDPL-GW inhibitor was increased via intranasal route (n=4-5). *p<0.05.

FIG. 6 shows that intranasal delivery of CDPL-GW nanoparticles abolishes chronic allergenic lung inflammation in the DRA model. Mice were subjected to the chronic DRA model, and then treated with CDPL-GW (1 ng/mouse via the i.n. route, twice a week) in three different designs as depicted in FIG. 14. CDPL-GW 3 wk indicates that the treatment with CDPL-GW was started at week 3 of the chronic DRA model and maintained until week 8 (total of 6 weeks). CDPL-GW Swk and 7 wk were done in a similar fashion. All of the measurements were done at week 11. (A) Treatment with CDPL-GW markedly reduced the DRA-induced BAL cellular recruitment which was mainly comprised of macrophages and lymphocytes. (B-E) CDPL-GW treatment starting at 3 and 5 weeks of the chronic DRA model (CDPL-GW 3 wk and Swk) effectively blocked the increases in total and DRA-reactive serum IgE, BAL IL-4 and IL-5, and attenuated lung inflammation and goblet cell metaplasia. However, the CDPL-GW 7 wk group showed similar trends, but differences failed to reach statistical significance in terms of DRA-reactive serum IgE, lung inflammation and goblet cell metaplasia. Scale bars represent 200 μm. (F) Single cell suspensions of regional LNs were re-stimulated with DRA for 72 hours. DRA re-stimulation increased the secretion of IL-4, IL-13 and IL-17, but CDPL-GW treatment blocked these increases. However, cytokine levels from cells obtained from the CDPL-GW 7 wk group did not reach statistical significance compared to those of the sham-treated DRA group, although they showed a declining trend. (G) Airway resistance was measured using increasing doses of methacholine in the sham and CDPL-GW treated groups after DRA challenge. All of the three CDPL-GW treated groups had significantly lower airway resistance compared to the sham treated chronic DRA group.

FIG. 7 shows (A) FACS strategy to identify alveolar cDC1 and cDC2 in BAL fluids. Lineage markers were defined in Methods. (B) In steady state, CSF1R⁺ cDCs were highly populated in the respiratory system, whereas the numbers of CSF1R⁺ cDCs were very low in bone marrow and blood. Of note, CSF1R⁺ cDC2s only emerged in BAL fluid, although CSF1R⁺ cDC1 resided highly both in the lung and BAL fluid.

FIG. 8 shows (A) All of the mice were challenged with DRA as depicted in the experimental scheme. For the depletion of the AEC-derived CSF1, Tm (75 mg/kg) was given by oral gavage to Scgb1a1-creERT; Csf1^{fi/fi} (CSF1^{ΔAEC}) mice. CSF1 (brown) is highly expressed in airway epithelial cells in DRA-challenged, but not sham-treated mice (Vehicle only). Administration of Tm depleted CSF1 expression, which was restored at 6 weeks after Tm administration. The contralateral lung was used for measuring CSF1 by ELISA. Tm treatment resulted in an immediate reduction in lung CSF1, which was restored to baseline by week 4. The scale bars represent 200 μm. *p<0.05.

FIG. 9 shows the docking pose of the compound GW2580. (A) Docking pose of GW2580 in the CSF1R kinase domain (PDB:4R7H). (B) Interactions of pivotal residues of CSF1R kinase domain with compound GW2580. Residues of the CSF1R kinase domain are colored in tan, while GW2580 is colored in cyan. Hydrogen bond (H-bond) interactions are in green dashed lines, while Pi-Pi interaction is a purple straight line. Two waters (red) play an important role in the H-bond interactions.

FIG. 10 shows CDPL-GW administered via intranasal route. In four hours, the intra-abdominal and thoracic images were simultaneously obtained by visible color imaging and dual-Near Infrared (NIR) channel FLARE™ imaging system. The organs were isolated from the mice and the images were taken. Signal to background ratio (SBR) was measured in each organ. *p<0.05.

FIG. 11 shows the size distribution of CDPL-GW nanoparticles as measured by NanoSight.

FIG. 12 shows the cellular distribution of CDPL-ZW by intranasal delivery. By tracing the fluorophore ZW800-1 of CDPL-GW, the distribution of CDPL-GW was evaluated by counting the fluorescent cells by flow cytometry. Each 1 ng and 100 ng of CDPL-GW per mouse was delivered via the intranasal route. In 2 hours, lung tissues were made into single cell suspensions. Single cell suspensions from the lung were gated into the positive ZW800, after which the cells were identified by cell specific markers.

FIG. 13 shows data for mice that were subject to the experiment scheme depicted in FIG. 5B. The concentrations of BAL protein and albumin were measured as the increasing doses of CDPL-GW were delivered via the intranasal route.

FIG. 14 shows the adverse effects of CDPL-ZW in the same experimental setting with FIG. 5B. CDPL-ZW did not significantly affect the apoptosis of alveolar macrophages and endothelial/epithelial which are represented by extremely elevarated BAL protein concentration and alumin amount, respectievely.

FIG. 15 shows experimental setting for FIG. 6. CDPL-GW intranasal treatment was started at three different time points, week 3, 5 and 7, in the chronic DRA model, during the repeated DRA exposures until week 8. After 3 weeks of rest, the mice were analyzed at week 11. Repeated treatment of CDPL-GW in chronic allergic inflammation did not affect the BAL concentration of total protein and albumin.

FIG. 16 shows that PLX3397 blocked allergen sensitization and attenuated subsequent allergic lung inflammation in the acute DRA induced murine model of asthma. A. SPR binding analysis for PLX3397. PLX3397 has a strong pharmacologic binding affinity to human recombinant CSF1 receptor with KD value of 5.31±0.51 nM. B. Experimental Design. DRA triple allergens were delivered via intranasal route as depicted. PLX3397 (10 mg/kg) was injected intraperitoneally on day 10, 12 and 14. C-E. In the acute DRA model, the treatment with PLX3397 blocked eosinophil recruitment to BAL fluid, attenuated serum IgE rise and Th2 cytokine increase in BAL fluids. F. Lung pathology revealed that PLX3397 treated group showed markedly reduced allergic lung inflammation.

Additional advantages of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### A. DEFINITIONS

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes.” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of”.

As used in the specification and the appended claims, the singular forms “a,” “an” and “the” include plural referents unless the context clearly dictates otherwise. Thus, for example, reference to “a CSF1R inhibitor,” “a pharmaceutical composition,” or “a microparticle,” includes, but is not limited to, two or more such CSF1R inhibitors, pharmaceutical compositions, or microparticles, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. “about x, y, z, or less” and should be interpreted to include the specific ranges of ‘about x’, ‘about y’, and ‘about z’ as well as the ranges of ‘less than x’ less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or ‘at or about’ whether or not expressly stated to be such. It is understood that where “about,” “approximate.” or “at or about” is used before a quantitative value, the parameter also includes the specific quantitative value itself, unless specifically stated otherwise.

“Microparticle” means a particle consisting of natural or synthetic polymers incorporating a small molecule CSF1R inhibitor and having particle diameters ranging from about 1 to about 1000 μm. The preparation of a wide variety of microparticles for drug delivery is known in the art.

“Modulating” or “modulate” refers to the treating, prevention, suppression, enhancement or induction of a function, condition or disorder. For example, it is believed that the compounds of the present disclosure can modulate asthma by interrupting the CSF1/CSF1R pathway by administration of small molecule inhibitors of CSF1R.

“Treating” or “treatment” as used herein covers the treatment of a disease or disorder described herein, in a subject, preferably a human, and includes: i. inhibiting a disease or disorder, i.e., (i) arresting its development; (ii) relieving a disease or disorder, such as causing regression of the disorder: (iii) slowing progression of the disorder: and/or (iv) inhibiting, relieving, or slowing the onset or progression of one or more symptoms of the disease or disorder.

“Subject” refers to a warm blooded animal such as a mammal, preferably a human, or a human child, which is afflicted with, or has the potential to be afflicted with one or more diseases and disorders described herein.

As used herein. “effective amount” can refer to the amount of a disclosed compound or pharmaceutical composition provided herein that is sufficient to effect beneficial or desired biological, emotional, medical, or clinical response of a cell, tissue, system, animal, or human.

An effective amount can be administered in one or more administrations, applications, or dosages. The term can also include within its scope amounts effective to enhance or restore to substantially normal physiological function.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration: the route of administration; the rate of excretion of the specific compound employed: the duration of the treatment: drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

The term “pharmaceutically acceptable vehicle” refers to a diluent, adjuvant, excipient or carrier with which a compound of the disclosure is administered. The terms “effective amount” or “pharmaceutically effective amount” refer to a nontoxic but sufficient amount of the agent to provide the desired biological result. That result can be reduction and/or alleviation of the signs, symptoms, or causes of a disease, or any other desired alteration of a biological system. An appropriate “effective” amount in any individual case can be determined by one of ordinary skill in the art using routine experimentation.

“Pharmaceutically acceptable carriers” for therapeutic use are well known in the pharmaceutical art, and are described, for example, in Remington's Pharmaceutical Sciences, 18th Edition (Easton, Pa.: Mack Publishing Company, 1990). For example, sterile saline and phosphate-buffered saline at physiological pH can be used. Preservatives, stabilizers, dyes and even flavoring agents can be provided in the pharmaceutical composition. For example, sodium benzoate, sorbic acid and esters of p-hydroxybenzoic acid can be added as preservatives. Id. at 1449. In addition, antioxidants and suspending agents can be used. Id.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable ester” refers to esters of compounds of the present disclosure which hydrolyze in vivo and include those that break down readily in the human body to leave the parent compound or a salt thereof. Examples of pharmaceutically acceptable, non-toxic esters of the present disclosure include C 1-to-C 6 alkyl esters and C 5-to-C 7 cycloalkyl esters, although C 1-to-C 4 alkyl esters are preferred. Esters of disclosed compounds can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, for example with methyl iodide, benzyl iodide, cyclopentyl iodide or alkyl triflate. They also can be prepared by reaction of the compound with an acid such as hydrochloric acid and an alcohol such as ethanol or methanol.

The term “pharmaceutically acceptable amide” refers to non-toxic amides of the present disclosure derived from ammonia, primary C 1-to-C 6 alkyl amines and secondary C 1-to-C 6 dialkyl amines. In the case of secondary amines, the amine can also be in the form of a 5- or 6-membered heterocycle containing one nitrogen atom. Amides derived from ammonia, C 1-to-C 3 alkyl primary amides and C 1-to-C 2 dialkyl secondary amides are preferred. Amides of disclosed compounds can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable amides are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, and piperidine. They also can be prepared by reaction of the compound with an acid such as sulfuric acid and an alkylcarboxylic acid such as acetic acid, or with acid and an arylcarboxylic acid such as benzoic acid under dehydrating conditions such as with molecular sieves added. The composition can contain a compound of the present disclosure in the form of a pharmaceutically acceptable prodrug.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

The term “contacting” as used herein refers to bringing a disclosed compound or pharmaceutical composition in proximity to a cell, a target protein, or other biological entity together in such a manner that the disclosed compound or pharmaceutical composition can affect the activity of the a cell, target protein, or other biological entity, either directly; i.e., by interacting with the cell, target protein, or other biological entity itself, or indirectly; i.e., by interacting with another molecule, co-factor, factor, or protein on which the activity of the cell, target protein, or other biological entity itself is dependent.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

As used herein, the terms “optional” or “optionally” means that the subsequently described event or circumstance can or cannot occur, and that the description includes instances where said event or circumstance occurs and instances where it does not.

It is understood, that unless otherwise specified, temperatures referred to herein are based on atmospheric pressure (i.e. one atmosphere).

In one aspect, the disclosure relates to methods of treating a disease or condition characterized by allergic inflammation, including, but not limited to, asthma, allergic conjunctivitis, allergic dermatitis, allergic esophagitis, allergic rhinitis, allergen-specific serum IgE production, allergic lung and airway inflammation and airway hyper-responsiveness (AHR) with minimal pulmonary adverse reaction, by administration of at least one CSF1R inhibitor and optionally a further therapeutic agent that is an anti-inflammatory drug and/or respiratory drug. In some aspects, the disclosed CSF1R inhibitors useful in the treatment of characterized by allergic inflammation can be administered using a disclosed pharmaceutical composition. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

As shown herein below, inhibition of the CSF1-CSF1R signaling pathway effectively suppresses sensitization to aeroallergens and consequent allergic lung inflammation in a murine model of chronic asthma. CDPL-GW nanoparticles showed favorable pharmacokinetics for inhalational treatment and intranasal insufflation delivery of CDPL-GW nanoparticles ameliorated asthma pathologies including allergen-specific serum IgE production, allergic lung and airway inflammation and airway hyper-responsiveness (AHR) with minimal pulmonary adverse reaction.

### B. CSF1R INHIBITOR COMPOUNDS

As used herein, “small molecule CSF1R inhibitor” means an organic compound having a molecular weight less than about 900 daltons, preferably less than about 500 daltons that interrupts the CSF1/CSF1R pathway by selectively blocking the CSF1 receptor CSF1R.

In further aspects, a CSF1R inhibitor can be, but is not limited to, AB-530, also known as N-[4-[3-(5-tert-Butyl-1,2-oxazol-3-yl)ureido]phenyl]imidazo[2,1-b]benzothiazole-2-carboxamide (Daiichi Sankyo), AC-708 (Ambit Bioscience), AC-710 (Ambit Bioscience). AC-855 (Ambit Bioscience). ARRY-382 (Array BioPharma), AZ-683 (Astra-Zeneca), AZD-6495 (Astra Zenenca), BLZ-3495 (Novartis), BLZ-945 (Novartis), N-(4-[[(5-tert-Butyl-1,2-oxazol-3-yl)carbamoyl]amino]phenyl)-5-[(1,2,2,6,6-pentamethylpiperidin-4-yl)oxy]pyrdine-2-carboxamide methanesulfonate (Daiichi Sankyo), N-(4-[[(5-tert-Butyl-1,2-oxazol-3-yl)carbamoyl]amino]phenyl)-5-[(1,2,2,6,6-pentamethylpiperidin-4-yl)oxy]pyridine-2-carboxamide methanesulfonate (Diaiichi Sankyo), CT 1578 (CTI BioPharma), CYT-645 (Gilead), DCC 2909 (Deciphera). DCC-3014 (Deciphera), DP-4577 (Deciphera), DP-5599 (Deciphera), DP-6261 (Deciphera), ENMD-981693 (EntreMed), FMS kinase inhibitors (AEgera), GT-79 (Gerinda Therapeutics), GW-2580 also known as 5-[3-Methoxy-4-(4-methoxybenzyloxy)benzyl]pyrimidine-2,4-diamine (GlaxoSmithKline), Ilorasertib (University of Chicago), Ki-20227 (Kyowa Hakko Kirin), Linifanib (AbbVie), Masitinib (AB Science). Pexidartinib (Plexxikon). PLX 5622 (Plexxikon). PLX FK1 (Plexxikon). PLX-7486 (Plexxikon), REDX-05182 (Redx Oncology), 5-cyano-N-[2-(cyclohexen-1-yl)-4-[1-[2-(dimethylamino)acetyl]piperidin-4-yl]phenyl]-1H-imidazole-2-carboxamide, 4-cyano-N-(2-(4,4-dimethylcyclohex-1-en-1-yl)-6-(2,2,6,6-tetramethyltetrahydro-2H-pyran-4-yl)pyridin-3-ly)-1H-imidazole-2-carboxamide or a solvate, hydrate, tautomer, or pharmaceutically acceptable salt thereof (Johnson & Johnson. Illig, C., et al., in US Patent Publication US2009/0105296 A1, published Apr. 23, 2009).

In a further aspect, the small molecule CSF-1R inhibitor is selected from the group consisting of AB-530, also known as N-[4-[3-(5-tert-Butyl-1,2-oxazol-3-yl)ureido]phenyl]imidazo[2,1-b]benzothiazole-2-carboxamide (Daiichi Sankyo). AC-708 (Ambit Bioscience), AC-710 (Ambit Bioscience), AC-855 (Ambit Bioscience), BLZ-3495 (Novartis). DCC-3014 (Deciphera), GW-2580 also known as 5-[3-Methoxy-4-(4-methoxybenzyloxy)benzyl]pyrimidine-2,4-diamine (GlaxoSmithKline), Ilorasertib (University of ChicagoMasitinib (AB Science). Pexidartinib (Plexxikon), PLX 5622 (Plexxikon). PLX FK1 (Plexxikon), PLX-7486 (Plexxikon), REDX-05182 (Redx Oncology), and 4-cyano-N-(2-(4,4-dimethylcyclohex-1-en-1-yl)-6-(2,2,6,6-tetramethyltetrahydro-2H-pyran-4-yl)pyridin-3-yl)-1H-imidazole-2-carboxamide or a solvate, hydrate, tautomer, or pharmaceutically acceptable salt thereof.

In a further aspect, a small molecule CSF1R inhibitors includes, but are not limited to PLX3397, DCC-3014, BLZ945, GW2580, PLX647 and ARRY-382.

“PLX3397” (Pexidartinib) means 5-((5-chloro-1H-pyrrolo[2,3-b]pyridin-3-yl)methyl)-N-((6-(trifluoromethyl)pyridin-3-yl)methyl)pyridin-2-amine. PLX3397 is commercially available, for example from Plexxikon Inc., Berkeley, Calif., and refers to the compound having a structure represented by the formula:

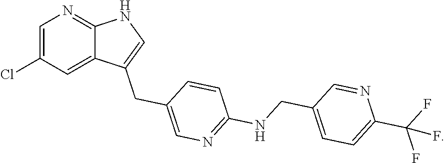 [SMILES: FC(F)(F)c1ccc(CNc2ccc(Cc3c[nH]c4ncc(Cl)cc34)cn2)cn1]

“DCC-3014” means the small molecule CSF1R inhibitor developed by Deciphera Pharmaceuticals, Inc., Waltham, Mass.

“BLZ945” means 4-[[2-[[(1R,2R)-2-hydroxycyclohexyl]amino]-6-benzothiazolyl]oxy]-N-methyl-2-pyridinecarboxamide. BLZ945 is commercially available, for example from Cayman Chemical. Ann Arbor, Mich., and refers to the compound having a structure represented by the formula:

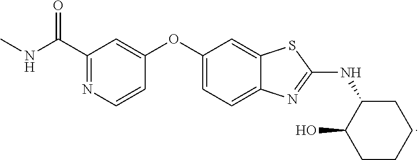 [SMILES: CNC(=O)c1cc(Oc2ccc3nc(N[C@@H]4CCCC[C@H]4O)sc3c2)ccn1]

“GW2580” means 5-[[3-Methoxy-4-[(4-methoxyphenyl)methoxy]phenyl]methyl]pyrimidine-2,4-diamine. GW2580 is commercially available, for example from Cayman Chemical, Ann Arbor, Mich., and refers to the compound having a structure represented by the formula:

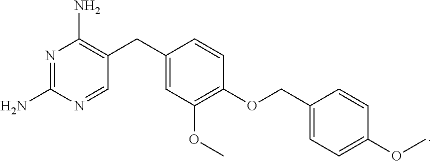 [SMILES: COc1ccc(COc2ccc(Cc3cnc(N)nc3N)cc2OC)cc1]

“PLX647” means 5-(1H-Pyrrolo[2,3-b]pyridin-3-ylmethyl)-N-[[4-(trifluoromethyl)phenyl]methyl]-2-pyridinamine. PLX647 is commercially available, for example from Cayman Chemical, Ann Arbor, Mich., and refers to the compound having a structure represented by the formula:

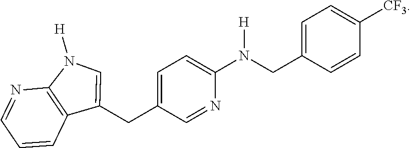 [SMILES: FC(F)(F)c1ccc(CNc2ccc(Cc3c[nH]c4ncccc34)cn2)cc1]

“ARRY-382” means the small molecule CSF1R inhibitor developed by Array Biopharma, Boulder, Colo.

In various aspects, the disclosed compounds can be in the form of a co-crystal. The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Preferred co-crystals include p-toluenesulfonic acid and benzenesulfonic acid.

The term “pharmaceutically acceptable co-crystal” means one that is compatible with the other ingredients of the formulation and not deleterious to the recipient thereof.

In a further aspect, the disclosed compounds can be isolated as solvates and, in particular, as hydrates of a disclosed compound, which can be obtained, for example, by crystallization from a solvent or from aqueous solution. In this connection, one, two, three or any arbitrary number of solvate or water molecules can combine with the compounds according to the invention to form solvates and hydrates.

The disclosed compounds can be used in the form of salts derived from inorganic or organic acids. Pharmaceutically acceptable salts include salts of acidic or basic groups present in the disclosed compounds. Suitable pharmaceutically acceptable salts include base addition salts, including alkali metal salts, e.g., sodium or potassium salts; alkaline earth metal salts, e.g., calcium or magnesium salts; and salts formed with suitable organic ligands, e.g., quaternary ammonium salts, which may be similarly prepared by reacting the drug compound with a suitable pharmaceutically acceptable base. The salts can be prepared in situ during the final isolation and purification of the compounds of the present disclosure; or following final isolation by reacting a free base function, such as a secondary or tertiary amine, of a disclosed compound with a suitable inorganic or organic acid; or reacting a free acid function, such as a carboxylic acid, of a disclosed compound with a suitable inorganic or organic base.

Acidic addition salts can be prepared in situ during the final isolation and purification of a disclosed compound, or separately by reacting moieties comprising one or more nitrogen groups with a suitable acid. In various aspects, acids which may be employed to form pharmaceutically acceptable acid addition salts include such inorganic acids as hydrochloric acid, sulphuric acid and phosphoric acid and such organic acids as oxalic acid, maleic acid, succinic acid and citric acid. In a further aspect, salts further include, but are not limited, to the following: hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate, butyrate, camphorate, camphorsulfonate, digluconate, glycerophosphate, hemisulfate, heptanoate, hexanoate, fumarate, hydrochloride, 2-hydroxyethanesulfonate (isethionate), nicotinate, 2-naphthalenesulfonate, oxalate, pectinate, persulfate, 3-phenylpropionate, picrate, pivalate, propionate, succinate, tartrate, thiocyanate, phosphate, glutamate, bicarbonate, undecanoate, and pamoate (i.e., 1,1′-methylene-bis-(2-hydroxy-3-naphthoate)) salts. Also, basic nitrogen-containing groups can be quaternized with such agents as lower alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others.

Basic addition salts can be prepared in situ during the final isolation and purification of a disclosed compound, or separately by reacting carboxylic acid moieties with a suitable base such as the hydroxide, carbonate or bicarbonate of a pharmaceutical acceptable metal cation or with ammonia, or an organic primary, secondary or tertiary amine. Pharmaceutical acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine and the like. In further aspects, bases which may be used in the preparation of pharmaceutically acceptable salts include the following: ammonia. L-arginine, benethamine, benzathine, calcium hydroxide, choline, deanol, diethanolamine, diethylamine, 2-(diethylamino)-ethanol, ethanolamine, ethylenediamine. N-methyl-glucamine, hydrabamine. 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, tiethanolamine, tromethamine and zinc hydroxide.

### C. PHARMACEUTICAL COMPOSITIONS

In certain aspects, this disclosure is a pharmaceutical composition comprising a therapeutically effective amount of one or more small molecule CSF1R inhibitors and one or more pharmaceutically acceptable diluents, preservatives, solubilizers, emulsifiers, adjuvants, excipients, or carriers. The pharmaceutical composition can be used, for example, for treating diseases or conditions characterized by allergic inflammation.

Suitable excipients for non-liquid formulations are also known to those of skill in the art. A thorough discussion of pharmaceutically acceptable excipients and salts is available in Remington's Pharmaceutical Sciences, 18th Edition (Easton, Pa.: Mack Publishing Company, 1990).

Additionally, auxiliary substances, such as wetting or emulsifying agents, biological buffering substances, surfactants, and the like, can be present in such vehicles. A biological buffer can be any solution which is pharmacologically acceptable and which provides the formulation with the desired pH. i.e., a pH in the physiologically acceptable range. Examples of buffer solutions include saline, phosphate buffered saline. Tris buffered saline, Hank's buffered saline, and the like.

Depending on the intended mode of administration, the pharmaceutical compositions can be in the form of solid, semi-solid or liquid dosage forms, such as, for example, tablets, suppositories, pills, capsules, powders, liquids, suspensions, creams, ointments, lotions or the like, preferably in unit dosage form suitable for single administration of a precise dosage. The compositions will include an effective amount of the selected drug in combination with a pharmaceutically acceptable carrier and, in addition, can include other pharmaceutical agents, adjuvants, diluents, buffers, and the like.

In general, the compositions of the disclosure will be administered in a therapeutically effective amount by any of the accepted modes of administration. Suitable dosage ranges depend upon numerous factors such as the severity of the disease to be treated, the age and relative health of the subject, the potency of the compound used, the route and form of administration, the indication towards which the administration is directed, and the preferences and experience of the medical practitioner involved. One of ordinary skill in the art of treating such diseases will be able, without undue experimentation and in reliance upon personal knowledge and the disclosure of this application, to ascertain a therapeutically effective amount of the compositions of the disclosure for a given disease.

Thus, the compositions of the disclosure can be administered as pharmaceutical formulations including those suitable for oral (including buccal and sub-lingual), rectal, nasal, topical, pulmonary, vaginal or parenteral (including intramuscular, intra-arterial, intrathecal, subcutaneous and intravenous) administration or in a form suitable for administration by inhalation or insufflation.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, and the like, an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered can also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, and the like. Actual methods of preparing such dosage forms are known, or will be apparent, to those skilled in this art; for example, see Remington's Pharmaceutical Sciences, referenced above.

Yet another aspect is the use of permeation enhancer excipients including polymers such as: polycations (chitosan and its quaternary ammonium derivatives, poly-L-arginine, aminated gelatin); polyanions (N-carboxymethyl chitosan, poly-acrylic acid); and, thiolated polymers (carboxymethyl cellulose-cysteine, polycarbophil-cysteine, chitosan-thiobutylamidine, chitosan-thioglycolic acid, chitosan-glutathione conjugates).

For oral administration, the composition will generally take the form of a tablet, capsule, a softgel capsule or can be an aqueous or nonaqueous solution, suspension or syrup. Tablets and capsules are preferred oral administration forms. Tablets and capsules for oral use can include one or more commonly used carriers such as lactose and corn starch. Lubricating agents, such as magnesium stearate, are also typically added. Typically, the compositions of the disclosure can be combined with an oral, non-toxic, pharmaceutically acceptable, inert carrier such as lactose, starch, sucrose, glucose, methyl callulose, magnesium stearate, dicalcium phosphate, calcium sulfate, mannitol, sorbitol and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethycellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

When liquid suspensions are used, the active agent can be combined with any oral, non-toxic, pharmaceutically acceptable inert carrier such as ethanol, glycerol, water, and the like and with emulsifying and suspending agents. If desired, flavoring, coloring and/or sweetening agents can be added as well. Other optional components for incorporation into an oral formulation herein include, but are not limited to, preservatives, suspending agents, thickening agents, and the like.

Parenteral formulations can be prepared in conventional forms, either as liquid solutions or suspensions, solid forms suitable for solubilization or suspension in liquid prior to injection, or as emulsions. Preferably, sterile injectable suspensions are formulated according to techniques known in the art using suitable carriers, dispersing or wetting agents and suspending agents. The sterile injectable formulation can also be a sterile injectable solution or a suspension in a nontoxic parenterally acceptable diluent or solvent. Among the acceptable vehicles and solvents that can be employed are water, Ringer's solution and isotonic sodium chloride solution. In addition, sterile, fixed oils, fatty esters or poyols are conventionally employed as solvents or suspending media. In addition, parenteral administration can involve the use of a slow release or sustained release system such that a constant level of dosage is maintained.

Parenteral administration includes intraarticular, intravenous, intramuscular, intradermal, intraperitoneal, and subcutaneous routes, and include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Administration via certain parenteral routes can involve introducing the formulations of the disclosure into the body of a patient through a needle or a catheter, propelled by a sterile syringe or some other mechanical device such as a continuous infusion system. A formulation provided by the disclosure can be administered using a syringe, injector, pump, or any other device recognized in the art for parenteral administration.

Preferably, sterile injectable suspensions are formulated according to techniques known in the art using suitable carriers, dispersing or wetting agents and suspending agents. The sterile injectable formulation can also be a sterile injectable solution or a suspension in a nontoxic parenterally acceptable diluent or solvent. Among the acceptable vehicles and solvents that can be employed are water, Ringer's solution and isotonic sodium chloride solution. In addition, sterile, fixed oils, fatty esters or polyols are conventionally employed as solvents or suspending media. In addition, parenteral administration can involve the use of a slow release or sustained release system such that a constant level of dosage is maintained.

Preparations according to the disclosure for parenteral administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms can also contain adjuvants such as preserving, wetting, emulsifying, and dispersing agents. They can be sterilized by, for example, filtration through a bacteria retaining filter, by incorporating sterilizing agents into the compositions, by irradiating the compositions, or by heating the compositions. They can also be manufactured using sterile water, or some other sterile injectable medium, immediately before use.

Sterile injectable solutions are prepared by incorporating one or more of the compounds of the disclosure in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Thus, for example, a parenteral composition suitable for administration by injection is prepared by stirring 1.5% by weight of active ingredient in 10% by volume propylene glycol and water. The solution is made isotonic with sodium chloride and sterilized.

Alternatively, the pharmaceutical compositions of the disclosure can be administered in the form of suppositories for rectal administration. These can be prepared by mixing the agent with a suitable nonirritating excipient which is solid at room temperature but liquid at the rectal temperature and therefore will melt in the rectum to release the drug. Such materials include cocoa butter, beeswax and polyethylene glycols.

Preferred formulations for topical drug delivery are ointments and creams. Ointments are semisolid preparations which are typically based on petrolatum or other petroleum derivatives. Creams containing the selected active agent, are, as known in the art, viscous liquid or semisolid emulsions, either oil-in-water or water-in-oil. Cream bases are water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. The specific ointment or cream base to be used, as will be appreciated by those skilled in the art, is one that will provide for optimum drug delivery. As with other carriers or vehicles, an ointment base should be inert, stable, nonirritating and nonsensitizing.

Formulations for buccal administration include tablets, lozenges, gels and the like. Alternatively, buccal administration can be effected using a transmucosal delivery system as known to those skilled in the art. The compounds of the disclosure can also be delivered through the skin or muscosal tissue using conventional transdermal drug delivery systems, i.e., transdermal “patches” wherein the agent is typically contained within a laminated structure that serves as a drug delivery device to be affixed to the body surface. In such a structure, the drug composition is typically contained in a layer, or “reservoir,” underlying an upper backing layer. The laminated device can contain a single reservoir, or it can contain multiple reservoirs. In one aspect, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, can be either a polymeric matrix as described above, or it can be a liquid or gel reservoir, or can take some other form. The backing layer in these laminates, which serves as the upper surface of the device, functions as the primary structural element of the laminated structure and provides the device with much of its flexibility. The material selected for the backing layer should be substantially impermeable to the active agent and any other materials that are present.

The pharmaceutical compositions of the disclosure can also be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, propellants such as fluorocarbons or nitrogen, and/or other conventional solubilizing or dispersing agents.

The compositions of the disclosure can be formulated for aerosol administration, particularly to the respiratory tract and including intranasal administration. Such compositions are prepared according to techniques well-known in the art of pharmaceutical formulation and can be prepared as solutions in saline, employing benzyl alcohol or other suitable preservatives, absorption promoters to enhance bioavailability, and/or other conventional solubilizing or dispersing agents. The compound will generally have a small particle size for example of the order of 5 microns or less. Such a particle size can be obtained by means known in the art, for example by micronization. The active ingredient is provided in a pressurized pack with a suitable propellant such as a chlorofluorocarbon (CFC) for example dichlorodifluoromethane, trichlorofluoromethane, or dichlorotetrafluoroethane, carbon dioxide or other suitable gas. The aerosol can conveniently also contain a surfactant such as lecithin. The dose of drug can be controlled by a metered valve. Alternatively the active ingredients can be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition can be presented in unit dose form for example in capsules or cartridges of e.g., gelatin or blister packs from which the powder can be administered by means of an inhaler.

The CSF1R inhibitor can be incorporated into opthalmic compositions suitable for administration to the subject. Typically, the ophthalmic composition or formulation comprises an opthalmic excipient. The ophthalmic excipient may be a buffer, tonicity adjuster, wetting agent, and/or an antioxidant. The buffer may be boric and/or phosphoric acid. The buffer may minimize changes to the pH of the nuclease composition. The tonicity adjuster may provide an isotonic environment and may include sodium chloride, postassium chloride, magnesium chloride, and/or boric acid. Antioxidants include sodiummetabisulfite and EDTA, for example. The antioxidants may be used to help stabilize the nuclease composition. Wetting agents, which include polyvinyl alcohol (PVA) and polysorbate 80, may allow the nuclease composition to spread over the eye. Other ophthalmic excipients include benzalkonium chloride (BAK), ethylenediaminetetraacetic acid (EDTA), purite, chlorobutanol, sodium perborate and sorbic acid, sodium perborate, purite, polyols, glycerin, polysorbate 80, dextran 70, propylene glycol, and polyethylene glycols, such as PEG-400. The ophthalmic excipient may be an ointment, such as mineral oil, white petrolatum, white ointment or lanolin. Similar to the aqueous vehicles, petrolatum and mineral oil may serve as vehicles in the ointment formulations to increase ocular contact time. These ingredients may help to form an occlusive film over the surface of the eyeball and improve the composition of the tear film by enhancing the mucin and aqueous layers. The ophthalmic excipient may provide mucin-like properties and/or decrease the loss of the aqueous layer due to evaporation. The ophthalmic exipient may function as a carrier, such as a pharmaceutically acceptable carrier as described below.

The opthalmic compositions may further comprise a pharmaceutically acceptable carrier suitable for ophthalmic delivery. Suitable ophthalmic carriers are known to those skilled in the art and all such conventional carriers may be employed in the present disclosure. Suitable carriers that may be used to facilitate and expedite transdermal delivery of topical compositions into ocular or adnexal tissues include, but are not limited to, alcohol (ethanol, propanol, and nonanol), fatty alcohol (lauryl alcohol), fatty acid (valeric acid, caproic acid and capric acid), fatty acid ester (isopropyl myristate and isopropyl n-hexanoate), alkyl ester (ethyl acetate and butyl acetate), polyol (propylene glycol, propanedione and hexanetriol), sulfoxide (dimethylsulfoxide and decylmethylsulfoxide), amide (urea, dimethylacetamide and pyrrolidone derivatives), surfactant (sodium lauryl sulfate, cetyltrimethylammonium bromide, polaxamers, spans, tweens, bile salts and lecithin), terpene (d-limonene, alpha-terpeneol. 1,8-cineole and menthone), and alkanone (N-heptane and N-nonane). Moreover, topically-administered compositions comprise surface adhesion molecule modulating agents including, but not limited to, a cadherin antagonist, a selectin antagonist, and an integrin antagonist. Optionally, the composition further contains a compound selected from the group consisting of a physiological acceptable salt, poloxamer analogs with carbopol, carbopohydroxypropyl methyl cellulose (HPMC), carbopol-methyl cellulose, carboxymethylcellulose (CMC), hyaluronic acid, cyclodextrin, and petroleum. Moreover, topically-administered compositions may comprise surface adhesion molecule modulating agents including, but not limited to, a cadherin antagonist, a selectin antagonist, and an integrin antagonist. Thus, a particular carrier may take the form of a sterile, ophthalmic ointment, cream, gel, solution, or dispersion. Also including as suitable ophthalmic carriers are slow release polymers, e.g., “Ocusert” polymers, “Hydron” polymers, etc.

Stabilizers may also be used such as, for example, chelating agents, e.g., EDTA. Antioxidants may also be used, e.g., sodium bisulfte, sodium thiosulfte. 8-hydroxy quinoline or ascorbic acid. Sterility typically will be maintained by conventional ophthalmic preservatives, e.g., chiorbutanol, benzalkonium chloride, cetylpyridium chloride, phenyl mercuric salts, thimerosal, etc., for aqueous formulations, and used in amounts which are nontoxic and which generally vary from about 0.001 to about 0.1% by weight of the aqueous solution. Conventional preservatives for ointments include methyl and propyl parabens. Typical ointment bases include white petrolatum and mineral oil or liquid petrolatum. However, preserved aqueous carriers are preferred. Solutions may be manually delivered to the eye in suitable dosage form. e.g., eye drops, or delivered by suitable microdrop or spray apparatus typically affording a metered dose of medicament. Examples of suitable ophthalmic carriers include sterile, substantially isotonic, aqueous solutions containing minor amounts, i.e., less than about 5% by weight hydroxypropylmethylcellulose, polyvinyl alcohol, carboxymethylcellulose, hydroxyethylcelullose, glycerine and EDTA. The solutions are preferably maintained at substantially neutral pH and isotonic with appropriate amounts of conventional buffers, e.g., phosphate, borate, acetate, tris.

Pharmaceutically acceptable ophthalmic carriers may further comprise amounts of auxiliary substances such as wetting or emulsifying agents, preservatives or buffers, which may enhance the shelf life or effectiveness of the nuclease, antibiotic compounds, anti-viral compounds, toll-like receptor antagonists, type-1 interferon antagonists, cathelicidin inhibitors, and/or a neutrophil elastase inhibitors.

A pharmaceutically or therapeutically effective amount of the composition comprising a CSF1R inhibitor will be delivered to the subject. The precise effective amount will vary from subject to subject and will depend upon the species, age, the subject's size and health, the nature and extent of the condition being treated, recommendations of the treating physician, and the therapeutics or combination of therapeutics selected for administration. Thus, the effective amount for a given situation can be determined by routine experimentation. The subject can be administered as many doses as is required to reduce and/or alleviate the signs, symptoms, or causes of the disorder in question, or bring about any other desired alteration of a biological system. When desired, formulations can be prepared with enteric coatings adapted for sustained or controlled release administration of the active ingredient.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The disclosed pharmaceutical compositions can comprise a disclosed CSF1R inhibitor and one or more additional therapeutic agents conventionally used to treat an airway inflammatory disorder such as an asthma pathology including, but not limited to, allergen-specific serum IgE production, allergic lung and airway inflammation and airway hyper-responsiveness (AHR) with minimal pulmonary adverse reaction. In a further aspect, the one or more therapeutic agents can be one or more of the following therapeutic agents used to treat an airway inflammatory disorder such as an asthma pathology including, but not limited to, allergen-specific serum IgE production, allergic lung and airway inflammation and airway hyper-responsiveness (AHR) with minimal pulmonary adverse reaction such a respiratory drug or an anti-inflammatory agent (e.g., corticosteroid, lipooxygenase inhibitor, mast cell stabilizer), including asthma inhibitors, asthma antagonists, and/or bronchodilators (e.g., a β-agonist). In a further aspect, the one or more therapeutic agent can be a corticosteroid, an anti IgE therapy, a j-adrenergic, a methylxanthine, an anticholinergics, corticosteroids, mediator-release inhibitors, anti-leukotriene drugs, anti-endothelin drugs, prostacyclin drugs, ion channel or pump inhibitors, enhancers, or modulators and pharmaceutically acceptable analogs, derivatives, and mixtures thereof. In other aspects, the one or more additional therapeutic agents can include, but is not limited to, may comprise Intal® (cromolyn) and/or Tilade@ (nedocromil), which help prevent asthma symptoms, especially symptoms caused by exercise, cold air and allergies.

Exemplary, but non-limiting examples of the one or more additional therapeutic agents include: (a) the corticosteroid can include, but is not limited to, cortisol, cortisone, hydrocortisone, fludrocortisone, prednisone, methylprednisonlone, or prednisolone etc: (b) the bronchodilator can include, but is not limited to, an anticholinergic, such as ipratropium or a beta-agonist such as albuterol, metaproterenol, pirbuterol, or levalbuteral; (c) an anti-IgE therapy can include Xolairg (omalizumab), which is approved for individuals with moderate to severe persistent asthma, year round allergies and who are taking routine inhaled steroids; (d) a leukotriene modifiers such as, for example, Accolate@ (zafirlukast), SingulairO (montelukast), and Zyflo® (zileuton).

In a further aspect, the one or more additional therapeutic agents can include espiratory drug is selected from the group consisting of albuterol, epinephrine, metaproterenol, terbutaline, pseudoephedrine hydrochloride, bambuterol, bitolterol, carbuterol, clenbuterol, clorprenalin, dioxethedrine, eprozinol, etefedrine, ethylnorepinephrine, fenoterol, fenspiride, hexoprenaline, isoetharine, isoproterenol, mabuterol, methoxyphenamine, pirbuterol, procaterol, protokylol, rimiterol, salmeterol, soterenol, tretoquinol, tulobuterol, caffeine, theophylline, aminophylline, acefylline, bamifylline, doxofylline, dyphylline, etamiphyllin, etofylline, proxyphylline, reproterol, theobromine-1-acetic acid, atropine, ipratropium bromide, flutropium bromide, oxitropium bromide, tiotropium bromide, budesonide, beclomethasone, ciclesonide, dexamethasone, flunisolide, fluticasone propionate, triamcinolone acetonide, prednisolone, methylprednisolone, hydrocortisone, cromolyn sodium, nedocromil sodium, montelukast, zafirukast, pirfenidone, CPX, IBMX, cilomilast, roflumilast, pumafentrine, domitroban, israpafant, ramatroban, seratrodast, tiaramide, zileuton, ambrisentan, bosentan, enrasentan, sitaxsentan, tezosentan, iloprost, treprostinil, and pharmaceutically acceptable analogs, derivatives, and mixtures thereof.

As used herein, the term “corticosteroid” refers to any of the adrenal corticosteroid hormones isolated from the adrenal cortex or produced synthetically, and derivatives thereof that are used for treatment of inflammatory diseases, such as arthritis, asthma, psoriasis, inflammatory bowel disease, lupus, and others. Corticosteroids include those that are naturally occurring, synthetic, or semi-synthetic in origin, and are characterized by the presence of a steroid nucleus of four fused rings, e.g., as found in cholesterol, dihydroxycholesterol, stigmasterol, and lanosterol structures. Corticosteroid drugs include cortisone, cortisol, hydrocortisone (11β,17-dihydroxy, 21-(phosphonooxy)-pregn-4-ene, 3,20-dione disodium), dihydroxycortisone, dexamethasone (21-(acetyloxy)-9-fluoro-11β,17-dihydroxy-16α-m-ethylpregna-1,4-diene-3,20-dione), and highly derivatized steroid drugs such as beconase (beclomethasone dipropionate, which is 9-chloro-11β, 17,21, trihydroxy-16β.-methylpregna-1,4 diene-3,20-dione 17,21-dipropionate). Other examples of corticosteroids include flunisolide, prednisone, prednisolone, methylprednisolone, triamcinolone, deflazacort and betamethasone.

Classes of bronchodilator drugs suitable for use with the described pharmaceutical compositions and treatment methods include the s-adrenergics, the methylxanthines, and the anticholinergics. Classes of anti-inflammatory drugs suitable for use with the described methods and devices include the corticosteroids, the mediator-release inhibitors, the anti-leukotriene drugs, as well as other inhibitors or antagonists. Other classes of respiratory drugs suitable for use with the described methods and devices include anti-endothelin drugs and prostacyclin drugs, which are particularly useful in the treatment of pulmonary fibrosis or hypertension, and ion channel or pump inhibitors, enhancers, and modulators, which are particulary useful in the treatment of cystic fibrosis. Exemplary s-adrenergics include, without limitation, albuterol, epinephrine, metaproterenol, terbutaline, pseudoephedrine hydrochloride, bambuterol, bitolterol, carbuterol, clenbuterol, clorprenalin, dioxethedrine, eprozinol, etefedrine, ethylnorepinephrine, fenoterol, fenspiride, hexoprenaline, isoetharine, isoproterenol, mabuterol, methoxyphenamine, pirbuterol, procaterol, protokylol, rimiterol, salmeterol, soterenol, tretoquinol, tulobuterol, and pharmaceutically acceptable analogs, derivatives, and mixtures thereof. Exemplary methylxanthines include, without limitation, caffeine, theophylline, aminophylline, acefylline, bamifylline, doxofylline, dyphylline, etamiphyllin, etofylline, proxyphylline, reproterol, theobromine-1-acetic acid, and pharmaceutically acceptable analogs, derivatives, and mixtures thereof. Exemplary anticholinergics include, without limitation, atropine, ipratropium bromide, flutropium bromide, oxitropium bromide, tiotropium bromide, and pharmaceutically acceptable analogs, derivatives, and mixtures thereof.

Exemplary corticosteroids include, without limitation, budesonide, beclomethasone, ciclesonide, dexamethasone, flunisolide, fluticasone propionate, triamcinolone acetonide, prednisolone, methylprednisolone, hydrocortisone, and pharmaceutically acceptable analogs, derivatives, and mixtures thereof. Exemplary mediator-release inhibitors include, without limitation, cromoyn sodium, nedocromil sodium, and pharmaceutically acceptable analogs, derivatives, and mixtures thereof. Exemplary anti-leukotrienes include, without limitation, montelukast, zafirukast, and pharmaceutically acceptable analogs, derivatives, and mixtures thereof. Other suitable respiratory drugs include, without limitation, pirfenidone, CPX, IBMX, cilomilast, roflumilast, pumafentrine, domitroban, israpafant, ramatroban, seratrodast, tiaramide, zileuton, ambrisentan, bosentan, enrasentan, sitaxsentan, tezosentan, iloprost, treprostinil, and pharmaceutically acceptable analogs, derivatives, and mixtures thereof.

### D. METHODS OF TREATING AN INFLAMMATORY DISORDER

Current treatments for allergic inflammation, including allergic asthma fall short of controlling pathology and clinical outcomes. A new approach targeting aeroallergen sensing in the early events of mucosal immunity could have greater benefit. The CSF1-CSF1R pathway has a critical role in trafficking allergens to regional lymph nodes through activating dendritic cells. Intervention in this pathway prevents allergen sensitization and subsequent Th2 allergic inflammation.

In one aspect, the disclosure relates to methods of treating an airway inflammatory disorder utilizing compounds that are CSF1R inhibitors. In some aspects, the airway inflammatory disorder can be an asthma pathology such as, but not limited to, allergen-specific serum IgE production, allergic lung and airway inflammation and airway hyper-responsiveness (AHR) with minimal pulmonary adverse reaction. In some aspects, the disclosed CSF1R inhibitors useful in the treatment of an airway inflammatory disorder are administered as a disclosed compound or a pharmaceutical composition, examples of which are discussed herein below.

### E. KITS

In a further aspect, the present disclosure relates to kits comprising at least one disclosed CSF1R inhibitor, or a pharmaceutically acceptable salt, hydrate, solvate, or polymorph thereof, and one or more of: (a) at least one agent known to treat a disorder associated with inflammation; (b) at least one agent known to treat allergic inflammation; (c) at least one agent known to treat a respiratory disease: (d) instructions for treating a disorder associated with inflammation; (e) instructions for treating allergic inflammation; or (f) instructions for administering the compound in connection with treating a respiratory disease.

The disclosed compounds and/or pharmaceutical compositions comprising the disclosed compounds can conveniently be presented as a kit, whereby two or more components, which may be active or inactive ingredients, carriers, diluents, and the like, are provided with instructions for preparation of the actual dosage form by the patient or person administering the drug to the patient. Such kits may be provided with all necessary materials and ingredients contained therein, or they may contain instructions for using or making materials or components that must be obtained independently by the patient or person administering the drug to the patient. In further aspects, a kit can include optional components that aid in the administration of the unit dose to patients, such as vials for reconstituting powder forms, syringes for injection, customized IV delivery systems, inhalers, etc. Additionally, a kit can contain instructions for preparation and administration of the compositions. The kit can be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

In a further aspect, the disclosed kits can be packaged in a daily dosing regimen (e.g., packaged on cards, packaged with dosing cards, packaged on blisters or blow-molded plastics, etc.). Such packaging promotes products and increases patient compliance with drug regimens. Such packaging can also reduce patient confusion. The present invention also features such kits further containing instructions for use.

In a further aspect, the present disclosure also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration.

In various aspects, the disclosed kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is contemplated that the disclosed kits can be used in connection with the disclosed methods of making, the disclosed methods of using or treating, and/or the disclosed compositions.

### F. REFERENCES

References are cited herein throughout using the format of reference number(s) enclosed by parentheses corresponding to one or more of the following numbered references.

For example, citation of references numbers 1 and 2 immediately herein below would be indicated in the disclosure as (1, 2).
1. Lambrecht B N, Hammad H. Biology of lung dendritic cells at the origin of asthma. Immunity 2009: 31: 412-424.
2. Hammad H, Lambrecht B N. Barrier Epithelial Cells and the Control of Type 2 Immunity. Immunity 2015; 43: 29-40.
3. Busse W W, Morgan W J, Gergen P J, Mitchell H E, Gern J E, Liu A H, Gruchalla R S. Kattan M, Teach S J, Pongracic J A. Chmiel J F, Steinbach S F, Calatroni A, Togias A, Thompson K M, Szefler S J, Sorkness C A. Randomized trial of omalizumab (anti-IgE) for asthma in inner-city children. N Engl J Med 2011; 364: 1005-1015.
4. Teach S J, Gill M A, Togias A, Sorkness C A, Arbes S J, Jr., Calatroni A, Wildfire J J, Gergen P J, Cohen R T, Pongracic J A. Kercsmar C M, Khurana Hershey G K, Gruchalla R S, Liu A H, Zoratti E M, Kattan M, Grindle K A. Gem J E, Busse W W. Szefier S J. Preseasonal treatment with either omalizumab or an inhaled corticosteroid boost to prevent fall asthma exacerbations. J Allergy Clin Immunol 2015; 136: 1476-1485.
5, Lambrecht B N, Hammad H. Allergens and the airway epithelium response: gateway to allergic sensitization. J Allergy Clin Immunol 2014; 134: 499-507.
6. Lee Y G, Jeong J J, Nyenhuis S, Berdyshev E, Chung S, Ranjan R, Karpurapu M, Deng J. Qian F. Kelly E A, Jarjour N N, Ackerman S J. Natarajan V. Christman J W, Park G Y. Recruited alveolar macrophages, in response to airway epithelial-derived monocyte chemoattractant protein 1/CCI2, regulate airway inflammation and remodeling in allergic asthma. Am J Respir Cell Mol Biol 2015; 52: 772-784.
7. Zaslona Z, Przybranowski S, Wilke C, van Rooijen N, Teitz-Tennenbaum S, Osterholzer J J, Wilkinson J E, Moore B B, Peters-Golden M. Resident alveolar macrophages suppress, whereas recruited monocytes promote, allergic lung inflammation in murine models of asthma. J Immunol 2014; 193: 4245-4253.
8. Joffre O, Nolte M A, Sporri R, Reis e Sousa C. Inflammatory signals in dendritic cell activation and the induction of adaptive immunity. Immunol Rev 2009; 227: 234-247.
9. Plantinga M, Guilliams M, Vanheerswynghels M, Deswarte K, Branco-Madeira F, Toussaint W. Vanhoutte L, Neyt K, Killeen N, Malissen B, Hammad H, Lambrecht B N. Conventional and monocyte-derived CD11b(+) dendritic cells initiate and maintain T helper 2 cell-mediated immunity to house dust mite allergen. Immunity 2013; 38: 322-335.
10. Moon H-G, Kim S-j, Jeong J J, Han S-S. Jarjour N N, Lee H, Abboud-Werner S L, Chung S, Choi H S, Natarajan V, Ackerman S J, Christman J W, Park G Y. Airway Epithelial Cell-Derived Colony Stimulating Factor-1 Promotes Allergen Sensitization. Immunity 2018; 49: 275-287.e275.
11. Goplen N. Karim M Z. Liang Q, Gorska M M, Rozario S, Guo L. Alam R. Combined sensitization of mice to extracts of dust mite, ragweed, and Aspergillus species breaks through tolerance and establishes chronic features of asthma. Journal of Allergy and Clinical Immunology 2009; 123: 925-932.e911.
12. Park G Y. Lee Y G, Berdyshev E. Nyenhuis S, Du J, Fu P, Gorshkova I A. Li Y, Chung S, Karpurapu M, Deng J, Ranjan R, Xiao L, Jaffe H A, Corbridge S J, Kelly E A, Jarjour N N, Chun J, Prestwich G D, Kaffe E, Ninou I, Aidinis V, Morris A J, Smyth S S, Ackerman S J, Natarajan V, Christman J W. Autotaxin production of lysophosphatidic acid mediates allergic asthmatic inflammation. Am J Respir Crit Care Med 2013; 188: 928-940.
13. Anderson D A, 3rd, Murphy K M, Briseno C G. Development, Diversity, and Function of Dendritic Cells in Mouse and Human. Cold Spring Harb Perspect Biol 2017.
14. Bajana S, Turner S, Paul J. Ainsua-Enrich E, Kovats S. IRF4 and IRF8 Act in CD11c+ Cells To Regulate Terminal Differentiation of Lung Tissue Dendritic Cells. J Immunol 2016; 196: 1666-1677.
15. Butowski N. Colman H, De Groot J F, Omuro A M, Nayak L. Wen P Y, Cloughesy T F, Marimuthu A, Haidar S, Perry A, Huse J, Phillips J, West B L, Nolop K B, Hsu H H, Ligon K L, Molinaro A M. Prados M. Orally administered colony stimulating factor 1 receptor inhibitor PLX3397 in recurrent glioblastoma: an Ivy Foundation Early Phase Clinical Trials Consortium phase II study. Neuro Oncol 2016: 18: 557-564.
16. Tap W D, Wainberg Z A. Anthony S P, Ibrahim P N, Zhang C. Healey J H, Chmielowski B, Staddon A P, Cohn A L, Shapiro G I, Keedy V L, Singh A S, Puzanov 1. Kwak E L. Wagner A J, Von Hoff D D. Weiss G J, Ramanathan R K, Zhang J, Habets G, Zhang Y, Burton E A, Visor G, Sanftner L, Severson P, Nguyen H, Kim M J, Marimuthu A, Tsang G, Shellooe R, Gee C, West B L, Hirth P, Nolop K, van de Rijn M, Hsu H H, Peterfy C. Lin P S. Tong-Starksen S, Bollag G. Structure-Guided Blockade of CSF1R Kinase in Tenosynovial Giant-Cell Tumor. N Engl J Med 2015; 373: 428-437.
17. Kang H. Gravier J, Bao K, Wada H. Lee J H. Baek Y, El Fakhri G, Gioux S, Rubin B P, Coll J L, Choi H S. Renal Clearable Organic Nanocariers for Bioimaging and Drug Delivery. Adv Mater 2016; 28: 8162-8168.
18. Choi H S. Ashitate Y, Lee J H, Kim S H, Matsui A, Insin N, Bawendi M G. Semmler-Behnke M, Frangioni J V, Tsuda A. Rapid translocation of nanoparticles from the lung airspaces to the body. Nat Biotechnol 2010; 28: 1300-1303.
19. Hose A J. Depner M, Illi S, Lau S, Keil T, Wahn U, Fuchs O, Pfeffere P I, Schmausser-Hechfelner E, Genuneit J, Lauener R, Karvonen A M, Roduit C, Dalphin J C, Riedler J. Pekkanen J, von Mutius E, Ege M J, Mas, groups Ps. Latent class analysis reveals clinically relevant atopy phenotypes in 2 birth cohorts. J Allergy Clin Immunol 2017; 139:1935-1945 e1912.
20. Rhodes H L, Thomas P, Sporik R, Holgate S T, Cogswell J J. A birth cohort study of subjects at risk of atopy: twenty-two-year follow-up of wheeze and atopic status. Am J Respir Crit Care Med 2002; 165: 176-180.
21. Ballardini N, Bergstrom A, Wahlgren C F, van Hage M, Hallner E. Kull I, Melen E, Anto J M, Bousquet J, Wickman M. IgE antibodies in relation to prevalence and multimorbidity of eczema, asthma, and rhinitis from birth to adolescence. Allergy 2016; 71: 342-349.
22. Durham S R, Emminger W, Kapp A, de Monchy J G, Rak S, Scadding G K, Wurtzen P A, Andersen J S, Tholstrup B, Riis B, Dahl R. S Q-standardized sublingual grass immunotherapy: confirmation of disease modification 2 years after 3 years of treatment in a randomized trial. J Allergy Clin Immunol 2012; 129: 717-725 e715.
23. Durham S R. Emminger W, Kapp A, Colombo G, de Monchy J G, Rak S. Scadding G K, Andersen J S, Riis B, Dahl R. Long-term clinical efficacy in grass pollen-induced rhinoconjunctivitis after treatment with S Q-standardized grass allergy immunotherapy tablet. J Allergy Clin Immunol 2010; 125: 131-138 e131-137.
24. Du Toit G, Roberts G, Sayre P H, Bahnson H T, Radulovic S, Santos A F, Brough H A, Phippard D. Basting M, Feeney M, Turcanu V. Sever M L, Gomez Lorenzo M, Plaut M, Lack G, Team L S. Randomized trial of peanut consumption in infants at risk for peanut allergy. N Engl J Med 2015; 372: 803-813.
25. Hammad H, Plantinga M, Deswarte K, Pouliot P, Willart M A, Kool M, Muskens F, Lambrecht B N. Inflammatory dendritic cells—not basophils—are necessary and sufficient for induction of Th2 immunity to inhaled house dust mite allergen. J Exp Med 2010; 207: 2097-2111.
26. Stanley E R, Chitu V. CSF-1 receptor signaling in myeloid cells. Cold Spring Harb Perspect Biol 2014; 6.
27. Turner S, Francis B, Vijverberg S, Pino-Yanes M, Maitland-van der Zee A H, Basu K, Bignell L, Mukhopadhyay S, Tavendale R, Palmer C, Hawcutt D, Pirmohamed M, Burchard E G. Lipworth B, Pharmacogenomics in Childhood Asthma C. Childhood asthma exacerbations and the Arg16 beta2-receptor polymorphism: A meta-analysis stratified by treatment. J Allergy Clin Immunol 2016; 138: 107-113 e105.
28. Zhu S, Chan-Yeung M, Becker A B, Dimich-Ward H, Ferguson A C, Manfreda J. Watson W T, Pare P D, Sandford A J. Polymorphisms of the IL-4, TNF-alpha, and Fcepsilon RIbeta genes and the risk of allergic disorders in at-risk infants. Am J Respir Crit Care Med 2000; 161: 1655-1659.
29. Sordillo J E, Kelly R, Bunyavanich S, McGeachie M. Qiu W. Croteau-Chonka D C, Soto-Quiros M, Avila L, Celedon J C, Brehm J M, Weiss S T, Gold D R, Litonjua A A. Genome-wide expression profiles identify potential targets for gene-environment interactions in asthma severity. J Allergy Clin Immunol 2015; 136: 885-892 e882.
30. Shin E K, Lee S H, Cho S H, Jung S, Yoon S H, Park S W, Park J S, Uh S T, Kim Y K, Kim Y H, Choi J S, Park B L, Shin H D, Park C S. Association between colony-stimulating factor 1 receptor gene polymorphisms and asthma risk. Hum Genet 2010; 128: 293-302.
31. Guilliams M, Scott C L. Does niche competition determine the origin of tissue-resident macrophages? Nat Rev Immunol 2017; 17: 451-460.
32. van de Laar L, Saelens W, De Prijck S, Martens L, Scott C L, Van lsterdael G, Hoffmann E, Beyaert R, Saeys Y, Lambrecht B N, Guilliams M. Yolk Sac Macrophages, Fetal Liver, and Adult Monocytes Can Colonize an Empty Niche and Develop into Functional Tissue-Resident Macrophages. Immunity 2016; 44: 755-768.
33. Moon H-G, Kim S-j, Jeong J J, Han S-S, Jarjour N N, Lee H, Abboud-Wemer S L, Chung S, Choi H S. Natarajan V, Ackerman S J, Christman J W, Park G Y. Airway Epithelial Cell-Derived Colony Stimulating Factor-1 Promotes Allergen Sensitization. Immunity 2018; 49: 275-287.e275.
34. Moon H G, Tae Y M, Kim Y S, Gyu Jeon S, Oh S Y, Song Gho Y, Zhu Z, Kim Y K. Conversion of Th17-type into Th2-type inflammation by acetyl salicylic acid via the adenosine and uric acid pathway in the lung. Allergy 2010: 65: 1093-1103.
35. Grozdanovic M, Laffey K G, Abdelkarim H, Hitchinson B, Harijith A, Moon H G, Park G Y, Rousslang L K, Masterson J C, Furuta G T, Tarasova N I, Gaponenko V, Ackerman S J. Novel peptide nanoparticle-biased antagonist of CCR3 blocks eosinophil recruitment and airway hyperresponsiveness. J Allergy Clin Immunol 2018.
36. Kang H, Gravier J, Bao K, Wada H, Lee J H, Baek Y, El Fakhri G, Gioux S, Rubin B P, Coll J L, Choi H S. Renal Clearable Organic Nanocariers for Bioimaging and Drug Delivery. Adv Mater 2016; 28: 8162-8168.
S5. Schrödinger Release 2016-1: Schrödinger Suite 2016-1 Protein Preparation Wizard; Epik version 3.5, Schrödinger. LLC, New York, N.Y.: Impact version 7.0, Schrödinger, LLC, New York. N Y; Prime version 4.3, Schrödinger, LLC, New York, N.Y. 2016.
37. Jorgensen W L, Maxwell D S. TiradoRives J. Development and testing of the OPLS all-atom force field on conformational energetics and properties of organic liquids. J Am Chem Soc 1996; 118: 11225-11236.
38. Schrödinger Release 2016-1: ligprep, version 3.7, Schrödinger, LLC, New York, N.Y. 2016.
39. Verdonk M L, Cole J C, Hartshom M J, Murray C W. Taylor R D. Improved protein-ligand docking using GOLD. Proteins: Structure. Function, and Bioinformatics 2003: 52: 609-623.
40. Korb O, Stützle T, Exner T E. Empirical Scoring Functions for Advanced Protein-Ligand Docking with PLANTS. Journal of Chemical Information and Modeling 2009; 49: 84-96.

### G. ASPECTS

The following listing of exemplary aspects supports and is supported by the disclosure provided herein.

Aspect 1. A method of treating a disease or condition characterized by allergic inflammation in a subject comprising administering to the subject an effective amount of a small molecule CSF1R inhibitor.

Aspect 2. The method of Aspect 1, wherein the CSF1R inhibitor is selected from PLX3397, DCC-3014, BLZ945, GW2580, PLX647 and ARRY-382, or a pharmaceutically acceptable salt thereof.

Aspect 3. The method of Aspect 2, wherein CSF1R inhibitor is GW2580 or PLX3397, or a pharmaceutically acceptable salt thereof.

Aspect 4. The method of any one of Aspects 1-3, wherein a microparticle comprising the small molecule CSF1R inhibitor is administered to the subject.

Aspect 5. The method of any one of Aspects 1-4, wherein the microparticle further comprises β-cyclodextrin conjugated epsilon-polylysine.

Aspect 6. The method of any one of Aspects 1-5, wherein the CSF1R inhibitor is administered to the patient during an acute allergic flare-up.

Aspect 7. The method of any one of Aspects 1-5, wherein the CSF1R inhibitor is administered to the patient prior to an acute allergic flare-up.

Aspect 8. The method of any one of Aspects 1-7, wherein the disease or condition characterized by allergic inflammation is selected from asthma, allergic conjunctivitis, allergic dermatitis, allergic esophagitis and allergic rhinitis.

Aspect 9. The method of Aspect 8, wherein the CSF1R inhibitor is GW2580 or PLX3397, or a pharmaceutically acceptable salt thereof.

Aspect 10. The method of Aspect 9, wherein the disease or condition characterized by allergic inflammation is asthma.

Aspect 11. The method of Aspect 10, wherein the CSF1R inhibitor is administered to the patient by nasal aerosol or inhalation.

Aspect 12. The method of Aspect 9, wherein the disease or condition characterized by allergic inflammation is allergic rhinitis.

Aspect 13. The method of Aspect 12, wherein the CSF1R inhibitor is administered to the patient by nasal aerosol or inhalation of a composition comprising the CSF1R inhibitor.

Aspect 14. The method of Aspect 9, wherein the disease or condition characterized by allergic inflammation is allergic conjunctivitis.

Aspect 15. The method of Aspect 14, wherein the CSF1R inhibitor is administered to the patient by application of an ophthalmic composition comprising the CSF1R inhibitor.

Aspect 16. The method of Aspect 9, wherein the disease or condition characterized by allergic inflammation is allergic dermatitis.

Aspect 17. The method of Aspect 16, wherein the CSF1R inhibitor is administered to the patient by application of a topical cream or ointment comprising the CSF1R inhibitor.

Aspect 18. The method of Aspect 9, wherein the disease or condition characterized by allergic inflammation is allergic esophagitis.

Aspect 19. The method of Aspect 18, wherein the CSF1R inhibitor is administered to the patient by ingestion of an oral composition comprising the CSF1R inhibitor.

Aspect 20. A method of modulating allergic inflammation in a subject exposed to sensitized allergen comprising administering to the subject an effective amount of a small molecule CSF1R inhibitor.

Aspect 21. The method of Aspect 20, wherein the CSF1R inhibitor is selected from PLX3397, DCC-3014, BLZ945, GW2580, PLX647 and ARRY-382, or a pharmaceutically acceptable salt thereof.

Aspect 22. The method of Aspect 21, wherein the CSF1R inhibitor is selected from PLX3397, DCC-3014, BLZ945, GW2580, PLX647 and ARRY-382, or a pharmaceutically acceptable salt thereof.

Aspect 23. The method of Aspect 21, wherein the CSF1R inhibitor is GW2580 or PLX3397, or a pharmaceutically acceptable salt thereof.

Aspect 24. The method of Aspect 21, wherein a microparticle comprising the small molecule CSF1R inhibitor is administered to the subject.

Aspect 25. The method of Aspect 24, wherein the nanoparticle further comprises μ-cyclodextrin conjugated epsilon-polylysine.

Aspect 26. The method of any one of Aspects 20-24, wherein the disease or condition characterized by allergic inflammation is selected from asthma, allergic conjunctivitis, allergic dermatitis, allergic esophagitis and allergic rhinitis.

Aspect 27. The method of Aspect 26, wherein the disease or condition characterized by allergic inflammation is asthma.

Aspect 28. The method of Aspect 27, wherein the CSF1R inhibitor is administered to the patient by nasal aerosol or inhalation of a composition comprising the CSF1R inhibitor.

Aspect 29. The method of any one of Aspects 20-24, wherein the disease or condition characterized by allergic inflammation is allergic rhinitis.

Aspect 30. The method of Aspect 29, wherein the CSF1R inhibitor is administered to the patient by nasal aerosol or inhalation of a composition comprising the CSF1R inhibitor.

Aspect 31. The method of any one of Aspects 20-24, wherein the disease or condition characterized by allergic inflammation is allergic conjunctivitis.

Aspect 32. The method of Aspect 31, wherein the CSF1R inhibitor is administered to the patient by application of an ophthalmic composition comprising the CSF1R inhibitor.

Aspect 33. The method of any one of Aspects 20-24, wherein the disease or condition characterized by allergic inflammation is allergic dermatitis.

Aspect 34. The method of Aspect 33, wherein the CSF1R inhibitor is administered to the patient by application of a topical cream or ointment comprising the CSF1R inhibitor.

Aspect 35. The method of any one of Aspects 20-24, wherein the disease or condition characterized by allergic inflammation is allergic esophagitis.

Aspect 36. The method of Aspect 35, wherein the CSF1R inhibitor is administered to the patient by ingestion of an oral composition comprising the CSF1R inhibitor.

Aspect 37. A microparticle comprising a CSF1R inhibitor or a pharmaceutically acceptable salt thereof.

Aspect 38. The microparticle of Aspect 37, wherein the CSF1R inhibitor is selected from PLX3397, DCC-3014, BLZ945, GW2580, PLX647 and ARRY-382, or a pharmaceutically acceptable salt thereof.

Aspect 39. The microparticle of Aspect 37 or 38, further comprising 1-cyclodextrin conjugated epsilon-polylysine.

Aspect 40. The microparticle of Aspect 39, wherein the CSF1R inhibitor is PLX3397 or GW2580, or a pharmaceutically acceptable salt thereof.

Aspect 41. An aerosol formulation comprising a CSF1R inhibitor is selected from PLX3397, DCC-3014, BLZ945, GW2580, PLX647 and ARRY-382, or a pharmaceutically acceptable salt thereof.

Aspect 42. An ophthalmic formulation comprising a CSF1R inhibitor is selected from PLX3397, DCC-3014, BLZ945, GW2580, PLX647 and ARRY-382, or a pharmaceutically acceptable salt thereof.

Aspect 43. A formulation for topical administration comprising a CSF1R inhibitor is selected from PLX3397, DCC-3014, BLZ945, GW2580, PLX647 and ARRY-382, or a pharmaceutically acceptable salt thereof and an ophthalmic excipient.

Aspect 44. A formulation for oral administration comprising a CSF1R inhibitor is selected from PLX3397, DCC-3014, BLZ945, GW2580, PLX647 and ARRY-382, or a pharmaceutically acceptable salt thereof.

From the foregoing, it will be seen that aspects herein are well adapted to attain all the ends and objects hereinabove set forth together with other advantages which are obvious and which are inherent to the structure.

While specific elements and steps are discussed in connection to one another, it is understood that any element and/or steps provided herein is contemplated as being combinable with any other elements and/or steps regardless of explicit provision of the same while still being within the scope provided herein.

It will be understood that certain features and subcombinations are of utility and may be employed without reference to other features and subcombinations. This is contemplated by and is within the scope of the claims.

Since many possible aspects may be made without departing from the scope thereof, it is to be understood that all matter herein set forth or shown in the accompanying drawings and detailed description is to be interpreted as illustrative and not in a limiting sense.

It is also to be understood that the terminology used herein is for the purpose of describing particular aspects only, and is not intended to be limiting. The skilled artisan will recognize many variants and adaptations of the aspects described herein. These variants and adaptations are intended to be included in the teachings of this disclosure and to be encompassed by the claims herein.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### H. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Mice. C57BL/6, Scgb1a1-CreERT (stock #016225), Irf4^{fi/fi} (stock #009380) and MAFIA (stock #005070) mice were purchased from Jackson Laboratory (Bar Harbor, Me.). Scgb1a1-creERT; Csf1^{fi/fi} and Csf1r-creERT; Irf4^{fi/fi} were generated as described previously (Moon H-G, et al. Immunity 2018 49:275-287.e275). Mice were bred in a specific pathogen-free facility maintained by the University of Illinois at Chicago. All mouse experiments were approved by the Institutional Animal Care and Use Committee of University of Illinois at Chicago. Age & sex-matched 7 to 10-week-old mice were used for experiments.

DRA-induced chronic asthma model. The previously described DRA-induced asthma model was used with minor modification (Goplen N, et al. J Allergy Clin Immunol 2009 123:925-932.e911). In brief, mice were subject to the DRA mixture comprised of house dust mite, ragweed and Aspergillus (5, 50, 5 μg/mouse, respectively) twice a week via intranasal route for 8 weeks. The mice were then rested for 3 weeks before collecting samples at week 11. To deplete the target genes in the experiments containing Scgb1a1-creERT; Csf1^{fi/fi} (CSF1^{ΔAEC}) and Csf1r-creERT; Irf4^{fi/fi} (IRF4^{ΔAPC}) strains, tamoxifen (Tm, 75 mg/kg, Sigma-Aldrich, St. Louis, Mo.) was administered via oral gavage for 5 consecutive days, according to the schedules depicted in Results.

Ex-vivo DRA antigen recall assay. LNs were obtained from the mediastinum of the mice subjected to the DRA-induced allergic lung models. A single cell suspension was made from the LNs and a total of 4×10⁶ cells/ml were plated in 24-well plates. For stimulation with the sensitizing allergens, the cells were treated with or without DRA (house dust mite, ragweed, and Aspergillus: 10, 100, 10 μg/ml, respectively) for 3 days. Cells were spun down and the supernatant used for ELISA.

Morphometric analysis and digital pathology. We used the Genie System (Aperio Technologies, Vista, Calif., US), a tool for automated classification and quantitation of tissue inflammation in whole slide images, as described previously (Park G Y, et al. Am J Respir Crit Care Med 2013 188:928-940). In this system, users identify the categories of tissue they want to quantify by outlining regions of interest that are examples of the tissue classes to be analyzed. Lung pathology was automatically quantified over entire lung fields by categorizing them into four types: areas of normal lung parenchyma, airway, void space (such as airway lumen) and inflammation. The pathologist doing the analysis was blinded to the experimental groups.

Statistical Analysis. Two-sample t test was used for two group comparisons and ANOVA was used for comparing multiple groups. The tests were two-sided and all data met the assumptions of the test. Each dot represents a single measurement of the parameters and the bar on the graph presents mean SEM. No statistical methods were used to predetermine sample sizes, but the distribution was assumed to be normal and variances were assumed to be equal across groups. All analyses were conducted using Prism. GraphPad Software (La Jolla, Calif.).

ELISA. Mouse CSF1 ELISA kit was purchased from R&D systems (Minneapolis, Minn.), mouse IL-4, IL-13, IL-17A, and IFN-γ ELISA kits were obtained from eBioscience (Waltham, Mass.) and anti-mouse IgE ELISA kit was from BioLegend (San Diego, Calif.). All procedures were followed by the manufacturer's instruction. For measuring DRA-reactive IgE, high affinity-binding 96-well plates were coated with DRA (D.f; 100, ragweed; 1000 and Aspergillus; 100 μg/ml, purchased from Greer Lab), in coating buffer for overnight at 4° C. Next, 1% bovine serum albumin (BSA) was used as blocking buffer for 1 h at room temperature and the rest of the procedure followed the IgE ELISA kit protocol as described previously (Moon H-G, et al. Immunity 2018 49:275-287.e275).

Flow cytometry. BAL cells, lung tissues, whole blood, and bone marrow were prepared for flow cytometry as described (Moon H G, et al. Allergy 2010 65:1093-1103). The lineage markers (Lin) containing anti-mouse CD3 (APC; BioLegend), anti-mouse B220 (APC; BioLegend), anti-mouse TER-119 (APC; BioLegend), anti-mouse F4/80 (APC; BioLegend) and anti-mouse CD64 (APC; BioLegend) were used to exclude T and B lymphocytes, erythrocytes and macrophages from the analysis. Anti-mouse CD11c (PE/Cy7; BioLegend), anti-mouse I-A/I-E (BV605; BioLegend), anti-mouse CD24 (BV510; BioLegend) and anti-mouse CD172a (AF700; BioLegend) used for cDC1 and cDC2 markers. Anti-GFP (AF488; BioLegend) and anti-mouse CSF1R (Percp; R&D systems) used for analyzing CSF1R expression in MAFIA mice. To identify the apoptosis, Annexin V apoptosis detection kit (BioLegend) was used by following the manufacturer's instruction. The analyses for conventional DC populations and CSF1R expression were done by using Kaluza software (Beckman Coulter, Indianapolis, Ind.).

Histology. Immunohistochemistry (IHC) & Periodic acid Schiff (PAS) staining. Formalin-fixed and paraffin-embedded sections of lung tissues were used for H&E, Immunohistochemistry (IHC), and Periodic acid-Schiff (PAS) staining. The primary antibodies used for the studies include anti-CSF1, anti-collagen, and anti-αSMA antibodies (Abam, Cambridge, Mass.). Histologic tissue processing and analyses were performed by Research Histology and Tissue Image Core (RHTIC) at UIC. All of the images were photographed using an Olympus BX51 fluorescence microscope (Olympus).

Measurement of Bronchial hyper-responsiveness. Mice were anesthetized with ketamine/xylazine mixture, and a tracheostomy tube was inserted. Mechanical ventilation was initiated using a Flexivent small animal ventilator (Sireq. Montreal. QC). Continuous EKG and pulse oximeter monitoring was performed. Airway resistance was measured after sequentially increasing doses of methacholine as described previously (Grozdanovic M, et al. J Allergy Clin Immunol 2018).

Synthesis of β-cyclodextrin (β-CD) conjugated epsilon-polylysine (CDPL). CDPL was prepared by a previous developed method (Kang H, et al. Adv Mater 2016 28:8162-8168). Briefly, to oxidize a hydroxyl group of β-cyclodextrin (β-CD), β-CD (1 g) and Dess-Martin periodinane (0.8 g) were dissolved in anhydrous DMSO (25 mL) and stirred for 12 h at room temperature. The reaction mixture was washed with cold acetone several times by precipitation method to remove insoluble impurities and the white solid dried in vacuum. An aldehyde-CD (700 mg) was dissolved in 25 mL of acetate buffer (0.2 M, pH 4.5) and then mixed with epsilon-polylysine (100 mg). After stirring for 1 h, sodium triacetoxyborohydride (263 mg) was added into the reaction mixture, followed by stirring for additional 72 h. Dialysis was carried out in cellulose membrane with a molecular weight cutoff (MWCO) of 12-14 kDa for 24 h against deionized water, and the resulting solution was freeze-dried. To visualize the CDPL nanoparticle, NIR fluorophore (ZW800-1C) was conjugated onto the CDPL chain using the conventional N-Hydroxysuccinimide (NHS) ester chemistry. Finally, succinic anhydride (half amount of the number of amines on CDPL chain) added into ZW800-CDPL in PBS (pH 8.0) to convert the surface charge from positive to zwitterionic and the resulting mixture was washed with acetone and dried in vacuum.

Preparation of GW2580-CDPL Inclusion complex. CDPL (2.5 μM in water) was mixed with GW2580 solution (20 μM in DMSO/PBS, 50/50 v/v %) and the mixture was vortexed for 24 h at room temperature. Then, the unbound GW2580 was removed by using micro Bio-Spin β-6 gel columns (Bio-rad). The absorbance of purified CDPL-GW solutions was then measured at around 280 nm to calculate the amount of GW2580.

In vivo bio-distribution and pharmacokinetics of GW2580-CDPL. Animals were housed in an AAALAC-certified facility and were studied under the supervision of Massachusetts General Hospital (MGH) Institutional Animal Care and Use Committee (IACUC) in accordance with the approved institutional protocol (#2016N000136). Six weeks old CD-1 mice (male; 25-30 g) were purchased from Charles River Laboratories (Wilmington, Mass.). Mice were maintained under anesthesia by inhalation of isoflurane or intraperitoneally injection with 100 mg/kg ketamine and 10 mg/kg xylazine (Webster Veterinary, Fort Devens, Mass.) The end of the tail was cut to be enable blood extraction. Before injection, blood was then sampled in heparinized capillary tubes (Fisher Scientific, Pittsburgh, Pa.) as a reference and collected blood was stored in an ice box to prevent clotting. Mice were injected with 10 nmol of CDPL-GW in saline and blood was collected to estimate blood half-life. Mice were imaged using the in-house built real-time intraoperative NIR imaging system. A 760 nm excitation laser source (4 mW/cm) was used with white light (400-650 nm; 40,000 lux). After 4 h post-injection, mice were sacrificed to evaluate the biodistribution of CDPL-GW in organs.

Molecular Docking Study of CSF1R kinase domain. The crystal structure of CSF1R Kinase was downloaded from the RCSB protein data bank and prepared by the Protein Preparation Wizard in Schrödinger 2016 (Schrödinger Release 2016-1: Schrödinger Suite 2016-1 Protein Preparation Wizard; Epik version 3.5. Schrödinger, LLC, New York, N.Y.; Impact version 7.0, Schrödinger. LLC, New York, N.Y.: Prime version 4.3. Schrödinger, LLC, New York, N.Y. 2016). During the protein preparation procedure, hydrogens were added and partial charges were assigned in the OPLS3 force field (Jorgensen W L. et al. J Am Chem Soc 1996 118:11225-11236), and then all the added hydrogens were minimized. The 3D structure of the GW2580 was processed by LigPrep module of Schrödinger 2016 (Schrödinger Release 2016-1: ligprep, version 3.7, Schrödinger, LLC, New York, N.Y. 2016) and then the GW2580 was docked into the above prepared CSF1R Kinase using GOLD v5.2.2 (Verdonk M L, et al. Proteins: Structure, Function, and Bioinformatics 2003 52:609-623). GW2580 was set to be flexible, while the CSF1R Kinase was set to be rigid. Standard default settings were used for other parameters. The binding poses for GW2580 were evaluated using the ChemPLP scoring function (Korb O, et al. Journal of Chemical Information and Modeling 2009 49:84-96) and the top scoring docking conformation was selected.

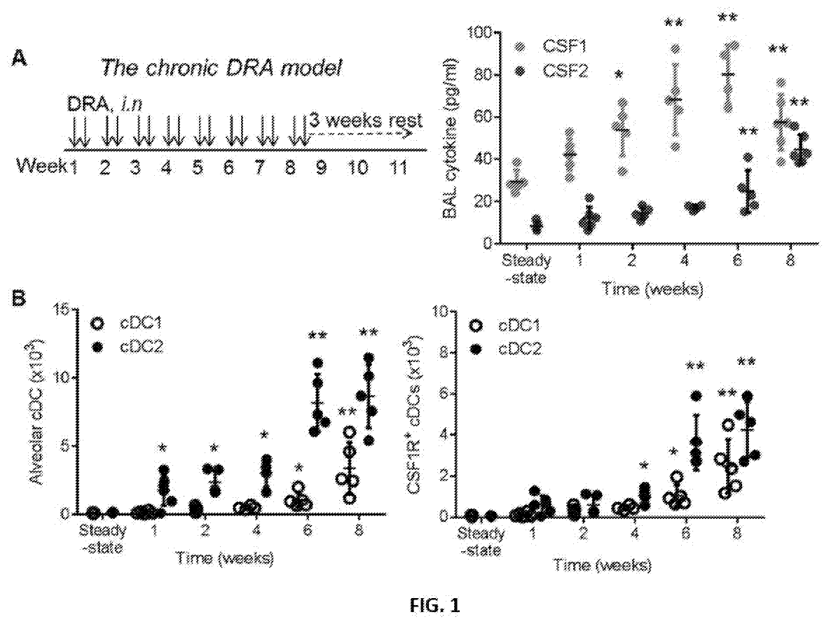

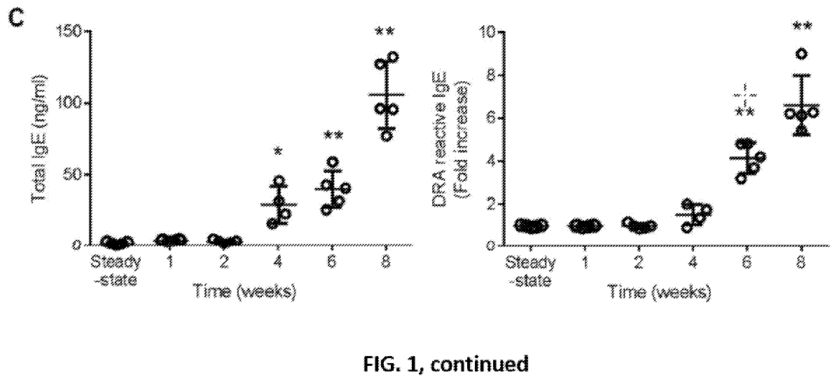

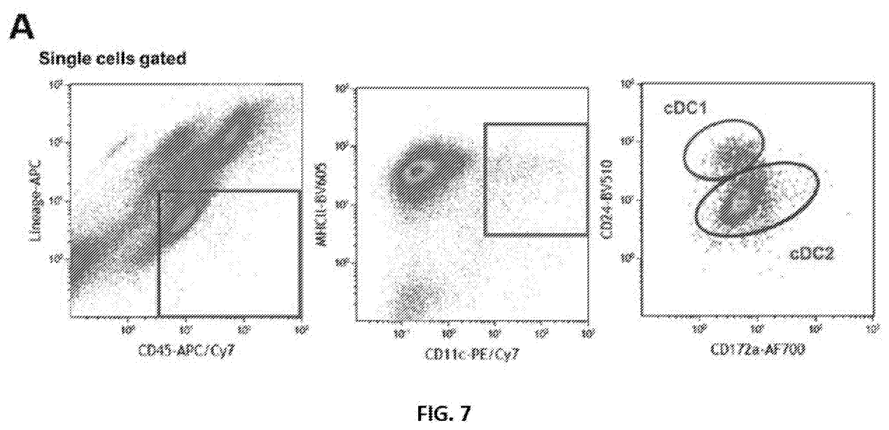

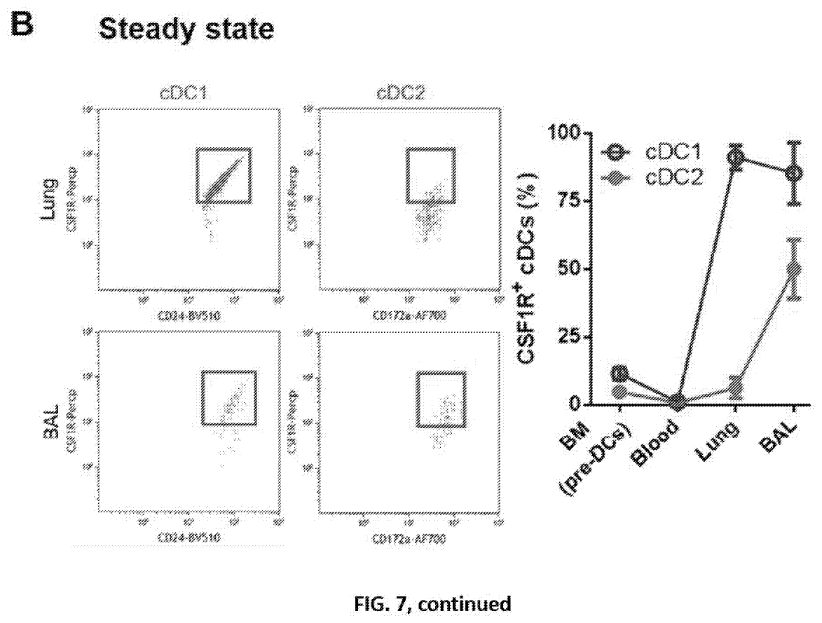

CSF1 and CSF1R⁺cDCs are highly enriched in the BAL fluid of the chronic DRA-induced murine model of asthma. To recapitulate a chronic inflammatory phenotype of human asthma, a chronic DRA-induced murine model of asthma was adopted (hereafter, the chronic DRA model) (FIG. 1A) (Moon H-G, et al. Immunity 2018 49:275-287.e275; Goplen N, et al. J Allergy Clin Immunol 2009 123:925-932.e911). Unlike the acute transient asthma models, eosinophilia in lung and BAL fluid was rarely seen in the chronic DRA model. Instead, the majority of BAL cells consisted of lymphocytes and macrophages and the characteristics of chronic airway inflammation and airway remodeling are well demonstrated in this model. Since AECs secrete CSF1 into the alveolar space in response to allergen exposure (Moon H-G, et al. Immunity 2018 49:275-287.e275), it was examined how chronic DRA exposure affects BAL CSF1 concentration. BAL CSF1 was promptly increased and peaked at week 6, whereas BAL CSF2 (GM-CSF) remained unchanged until week 6, when it began to rise (FIG. 1A). In steady state, cDCs were highly populated in the respiratory system, whereas their numbers were low in blood (FIG. 7). Next, mice were subject to the chronic DRA model and cDCs in BAL were measured by using the flow cytometric gating as depicted in FIG. 7. Similar to the pattern of BAL CSF1, cDC2s were rapidly increased over the course of the chronic DRA model, but cDC1s were delayed until week 6. Because CSF1R⁺ cDC2 play critical role in allergen sensitization (Moon H-G, et al. Immunity 2018 49:275-287.e275), we further analyzed CSF1R⁺ cDCs in the BAL fluids. Chronic DRA exposure induced an earlier and higher increase in CSF1R⁺ cDC2 than that of CSF1R⁺ cDC1 (FIG. 1B). Total and DRA-reactive serum IgE levels were also progressively increased, indicating that allergen sensitization has been intensified by repeated exposure to the allergens (FIG. 1C).

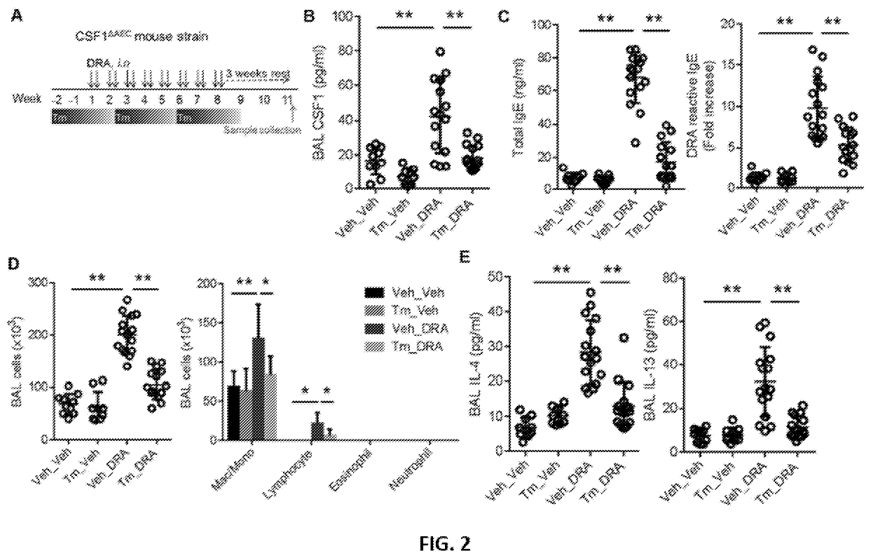

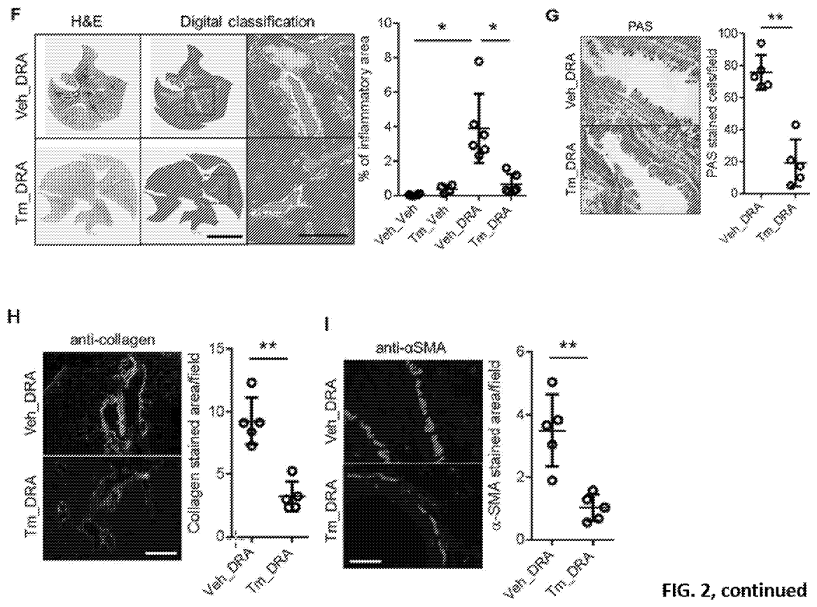

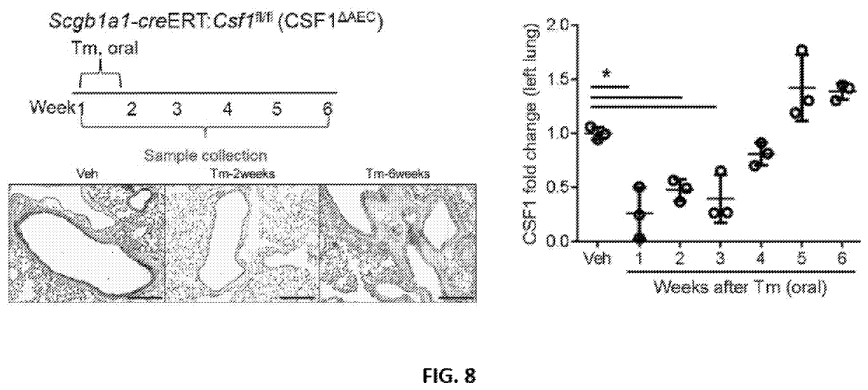

AEC-derived CSF1 is necessary for development of chronic allergic lung inflammation and airway remodeling. To test whether CSF1 secreted by AECs is required for establishing chronic allergic inflammation and airway remodeling, the Scgb1a1-creERT:Csf1^{fi/fi} (hereafter CSF1^{ΔAEC}) mice was used which are the inducible knock-out mice of CSF1 selectively in AECs by tamoxifen (Tm) injection (Moon H-G, et al. Immunity 2018 49:275-287.e275). First, the duration of CSF1 depletion was measured by one single cycle (consisting of 5 consecutive days) of Tm ingestion. The CSF1 depletion in AECs and BAL fluids continued up to 4 weeks by one cycle of oral Tm (FIG. 8). Therefore, tamoxifen was administered every 4 weeks during the chronic DRA model (FIG. 2A). CSF1 in BAL was increased about 2-3 fold in the chronic DRA group (Veh_DRA), compared to the sham-treated or only tamoxifen groups (Veh_Veh, Tm_Veh), but tamoxifen treatment abolished the increase of CSF1 (Tm_DRA) (FIG. 2B). CSF1 has a critical role in allergen sensitization and IgE production by facilitating DC mobilization (Moon H-G, et al. Immunity 2018 49:275-287.e275). In the chronic DRA model, blocking the AEC secretion of CSF1 significantly reduced total and DRA-reactive serum IgE production (FIG. 2C) and abrogated the subsequent allergic lung inflammation, inflammatory cell infiltration, Th2 cytokine production and airway remodeling, including goblet cell metaplasia, collagen deposition and smooth muscle hypertrophy (FIG. 2D-1).

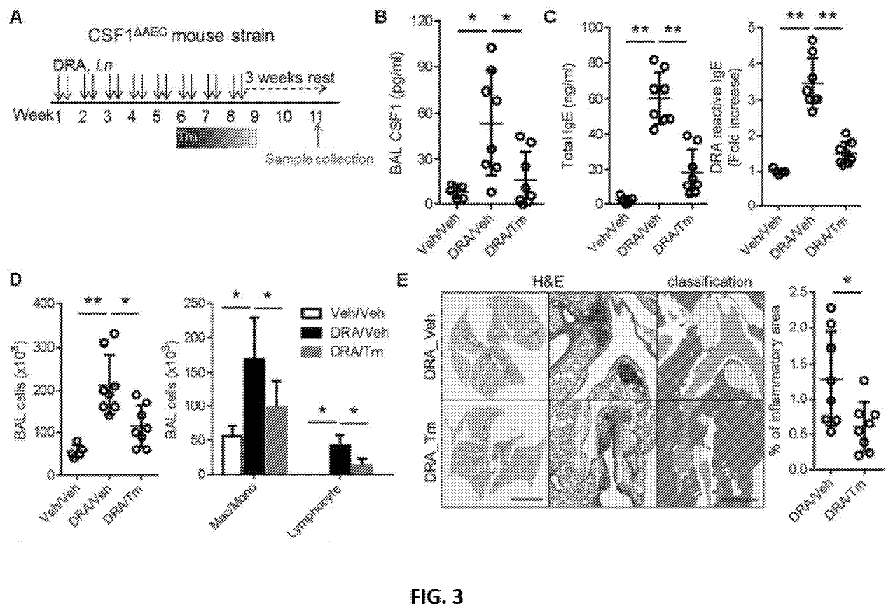

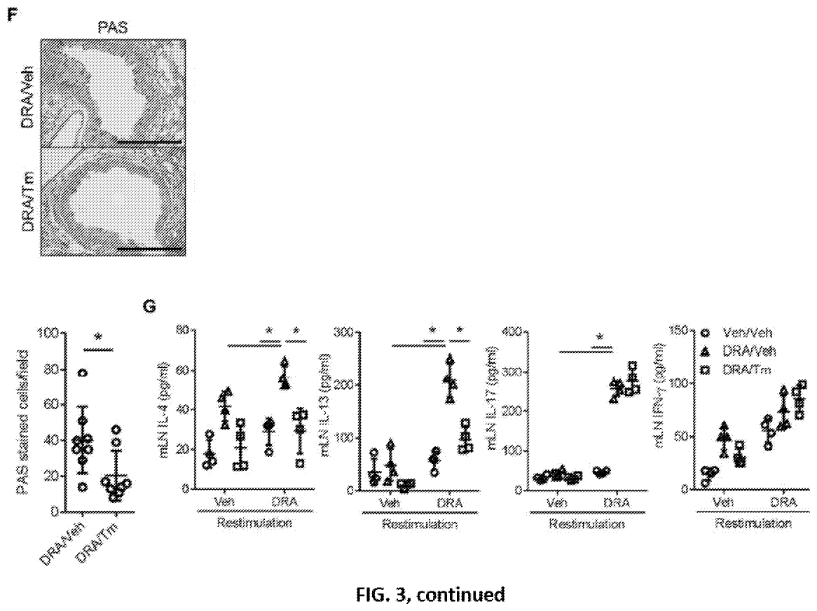

CSF1 depletion reverses established chronic allergic inflammation and airway remodeling, and suppresses Th2 memory responses. To ensure relevance to treatment of human asthma, a model in which CSF1 depletion took place after the allergic lung inflammation had been fully established (FIG. 3A) was created. In this model, oral Tm ingestion was given for one cycle at week 6 of the chronic DRA model. The one cycle of oral Tm treatment resulted in significantly decreased BAL CSF1 (FIG. 3B). The late depletion of CSF1 in the chronic DRA model was able to reverse the allergen sensitization (serum IgE), inflammatory cell infiltration, allergic lung inflammation and goblet cell metaplasia (FIG. 3C-F). A single cell suspension was made from the LNs obtained from the mice subjected to the above model and used for the ex-vivo DRA antigen recall assay as described in Methods. IL-4 and IL-13 secretion were boosted by re-challenge with DRA, but blunted in the cells obtained from the CSF1-depleted mice (FIG. 3G), indicating that the Th2 memory response was significantly reduced in CSF1-depleted mice. Of interest, depletion of CSF1 had no effect on IL-17 and IFN-γ secretion by DRA re-stimulation, suggesting that CSF1 influence is selective for the Th2 immune response.

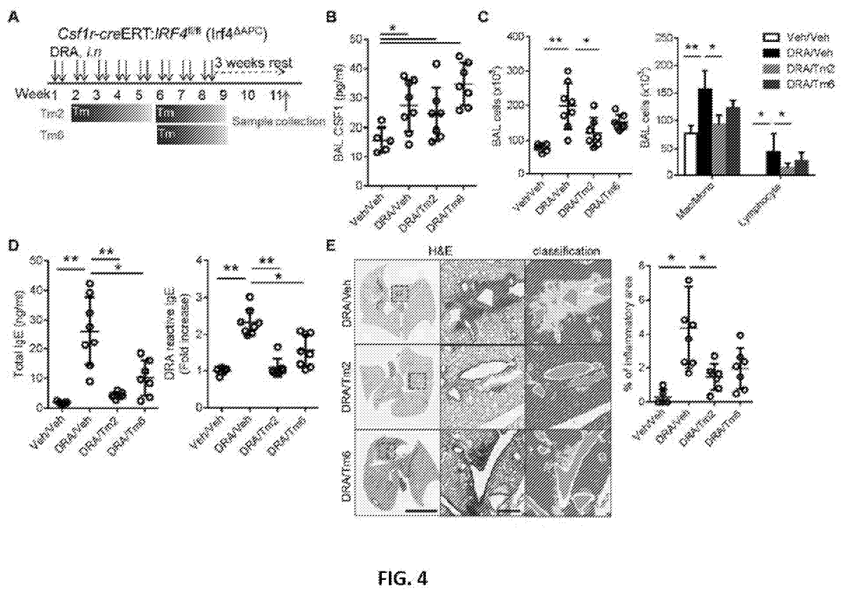

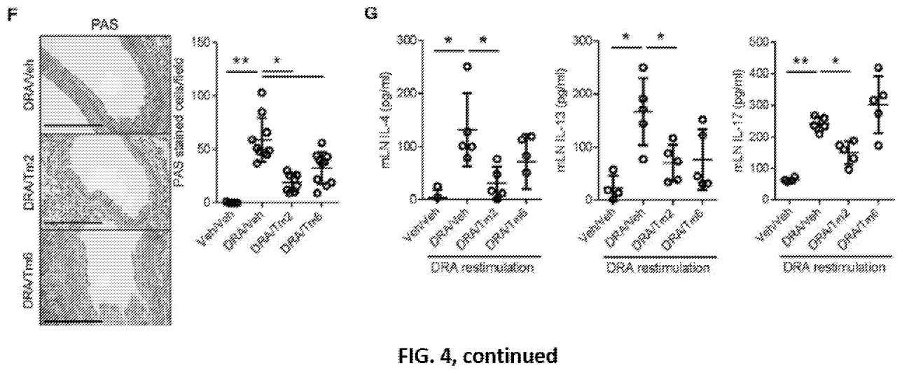

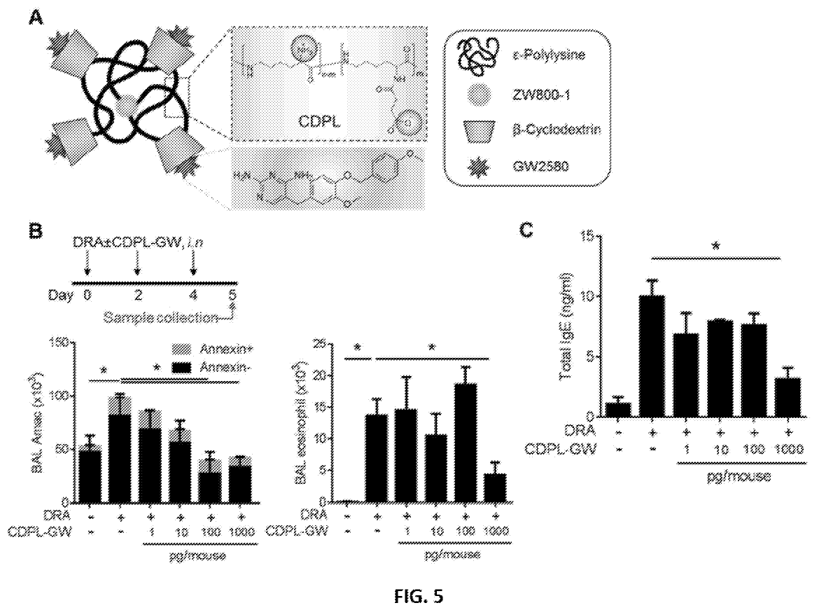

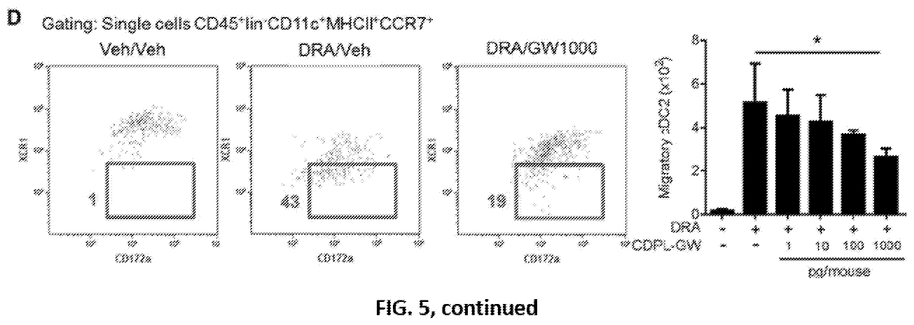

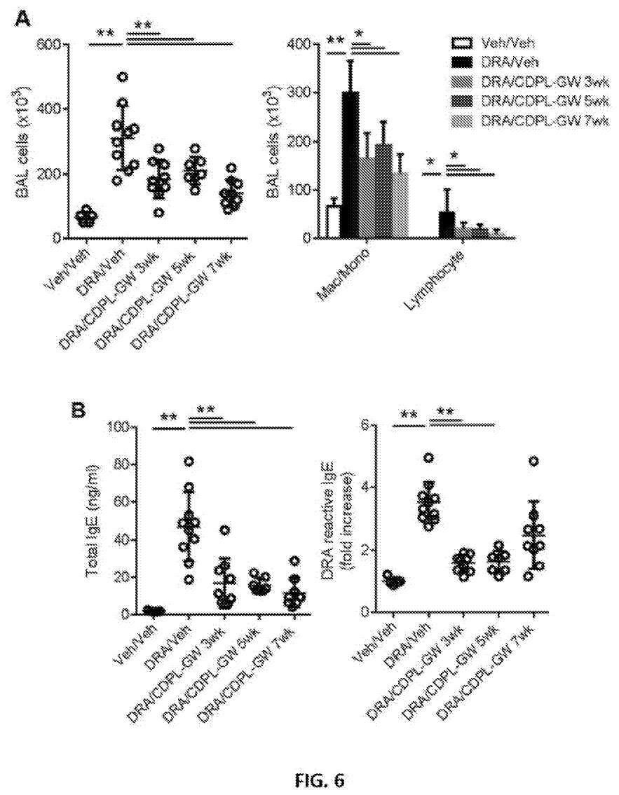

IRF4′ CSF1R′ cells are required for Th2 memory in the secondary LNs of the chronic DRA model, cDC2 play a critical role in CSF1-dependent allergen sensitization (Moon H-G, et al. Immunity 2018 49:275-287.e275). Since there is no mouse strain in which cDC2 development is selectively ablated (Anderson D A, 3rd, Murphy K M, Briseno C G, Development, Diversity, and Function of Dendritic Cells in Mouse and Human. Cold Spring Harb Perspect Biol 2017), we used Csf1r-creERT; Irf4^{fi/fi} (hereafter IRF4^{ΔAPC}) for a cDC2 targeted experiment (Moon H-G, et al. Immunity 2018 49:275-287.e275). Because IRF4 is required for cDC2 development and function, but not for cDC1 (Bajana S, Tet al. J Immunol 2016; 196:1666-1677), and Csf1r expressing macrophages are not engaged in antigen trafficking to LNs, this mouse could be used for targeting cDC2 function in the experimental model of allergen sensitization (Moon H-G, et al. Immunity 2018 49:275-287.e275). The mice were subjected to the chronic DRA model as depicted in FIG. 4A. In these two models, the allergic inflammation was established first, and then Tm administered to deplete the CSF1R⁺ IRF4⁺ cells at either week 2 (Tm2) or 6 (Tm6). Despite the presence of sufficient CSF1 in BAL fluid (FIG. 4B), the depletion of CSF1R⁺IRF4⁺ cells resulted in reduction of inflammatory cell infiltration and total and DRA-reactive serum IgE production, and the attenuation of lung inflammation and goblet cell metaplasia, although the Tm6 group showed only modest effects (FIG. 4C-F). The ex-vivo DRA antigen recall assay showed that the depletion of CSF1R⁺IRF4⁺ blocked the production of IL-4 and IL-13 by DRA re-stimulation in both Tm2 and Tm6 treatment groups, whereas IL-17 production remained intact in the Tm6 group, while samples from the Tm2 group showed partial reduction (FIG. 4G). Collectively, these data indicate that CSF1R*cDC2 are required for the formation of Th2 memory cells, indicating that interfering with cDC2 function should be beneficial for treating Th2-immune mediated diseases.

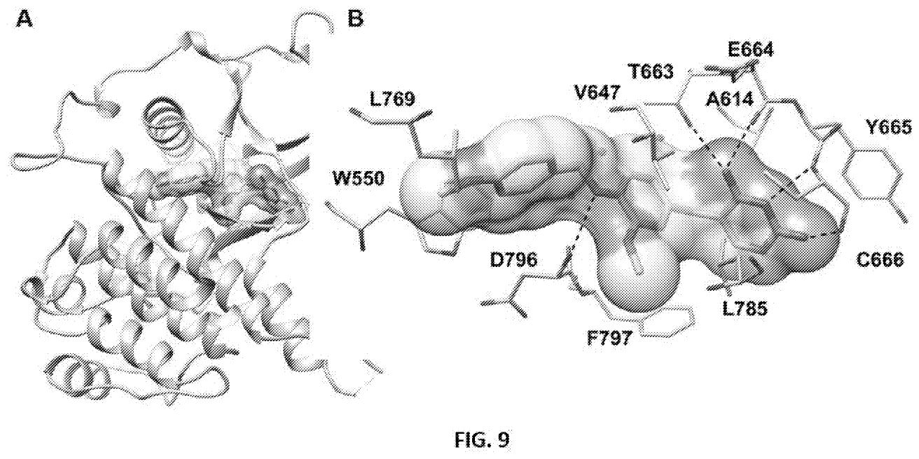

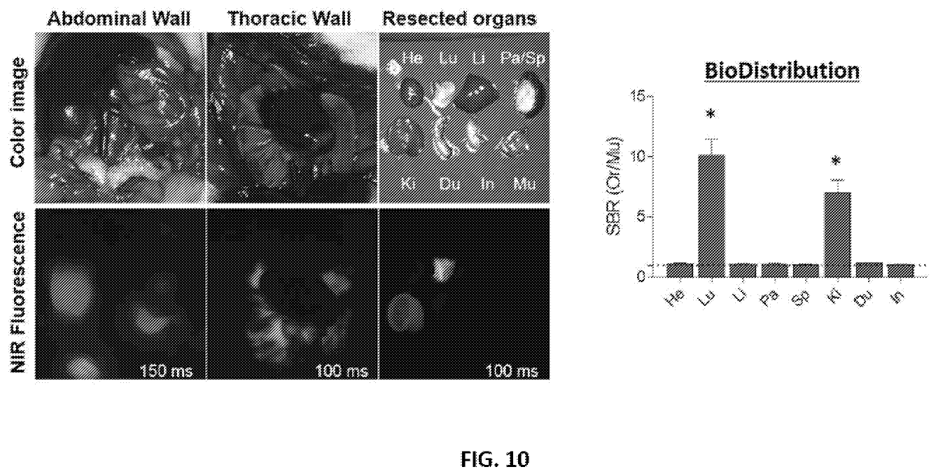

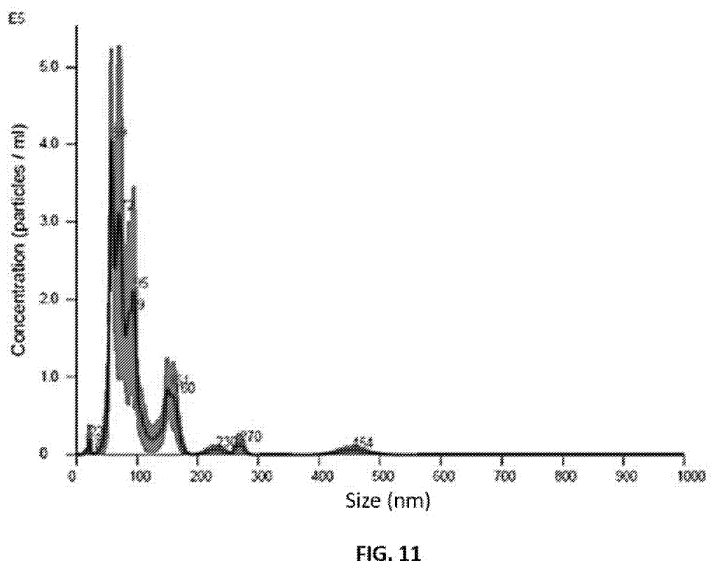

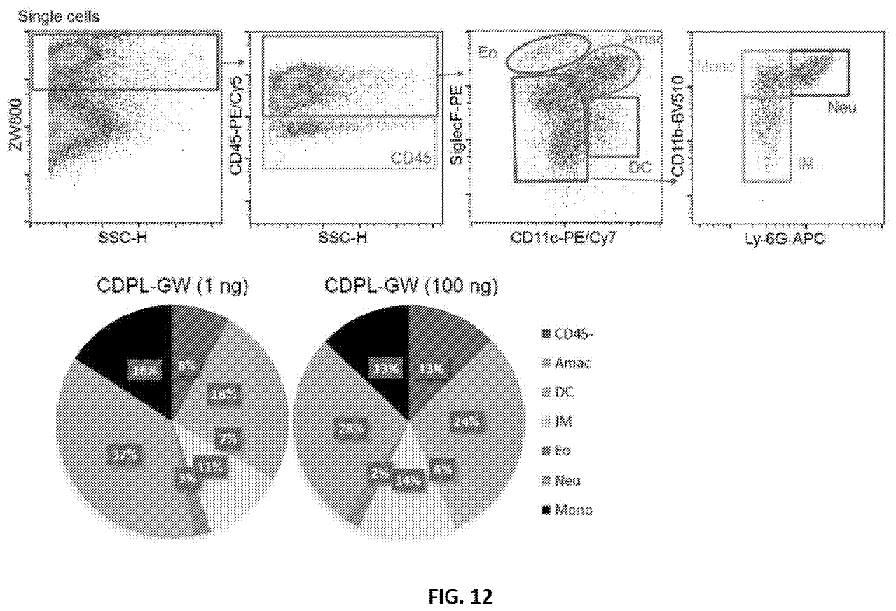

Nanoparticles carrying a CSF1R inhibitor blocks cDC2 migration and allergen sensitization. Selective CSF1R inhibitors have been used in continuing clinical trials for cancer treatment with minimal adverse effects (Butowski N, et al. Neuro Oncol 201618:557-564; Tap W D, et al. N Engl J Med 2015 373:428-437). GW2580, a CSF1R inhibitor, showed excellent binding affinity to human CSF1R recombinant protein (Moon H-G, et al. Immunity 2018 49:275-287.e275). Simulation protein structure modeling indicated that GW2580 interacts with two critical intracellular tyrosine residues (Y546 and Y665) of the kinase domain of CSF1R (FIG. 9). Further, systemic administration of GW 2580 effectively blocked cDC2 migration to regional LNs (Moon H-G, et al. Immunity 2018 49:275-287.e275). The inhalational route is preferred for asthma treatment because it can deliver a compound effectively to the target organ and minimize systemic adverse effects. For an inhalational delivery, a CPDL-encapsulated nanoparticle containing CSF1R inhibitor was generated using a previously described method (Kang H, et al. Adv Mater 2016 28:8162-8168). CDPL nanoparticles increase the chemical stability and bioavailability of guest molecules to ensure site-specific delivery of lipophilic GW2580 to the lung without sticking to other tissues (Choi H S. et al. Nat Biotechnol 2010 28:1300-1303). Each CPDL nanoparticle carries four molecules of GW2580 as well as a ZW800-1 fluorophore (CDPL-GW) (FIG. 5A). As expected, intranasal delivery of CDPL-GW had good lung deposition (FIG. 10). Measurements by NanoSight (Solisbury, U.K.) indicated that most of the CPDL nanoparticles were less than 100 nm (FIG. 11). Next, the cellular distribution of two different doses of CDPL-GW (1 ng and 100 ng/mouse) was analyzed via intranasal insufflation to the lung (FIG. 12). Neutrophils. DCs and alveolar macrophages (AMs) were the major cells that took up the particles. To optimize the dose of CDPL-GW for inhibiting DC migration in lung, the mice were subjected to DRA challenge for 5 days with increasing dose of CDPL-GW (from 1 pg to 1 ng/mouse) and the samples were collected on day 5 (FIG. 5B). Total BAL cells were increased by DRA challenge, but suppressed by CDPL-GW in a dose-dependent manner (FIG. 5B), and BAL eosinophil counts and serum IgE concentrations were also markedly reduced by 1 ng/mouse of CDPL-GW (FIG. 5B-C).

However, there was no significant apoptotic death of AMs or increases in BAL total protein or albumin concentration by CDPL-GW treatment (FIGS. 5B & 13). To further characterize the effects of CDPL-GW on DCs, the number of migratory DCs (CD45⁺lin·CD11c⁺MHCII⁺CCR7⁺) was evaluated in regional LNs. CDPL-GW suppressed migratory cDC2 in a dose-dependent manner (FIG. 5D). From these data, it was determined that 1 ng of CDPL-GW/mouse (equivalent to 8000 nanoparticles) as the therapeutic dose for the next experiment.

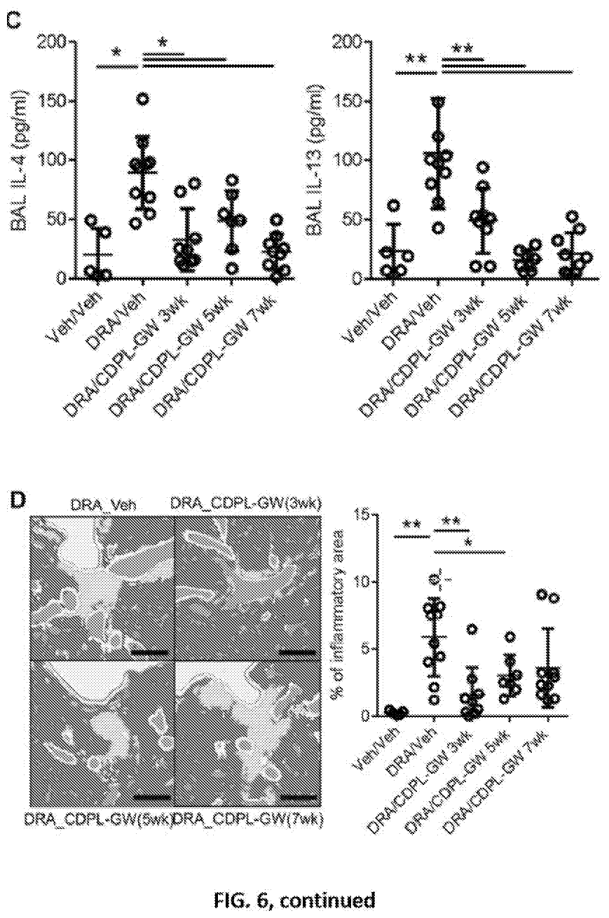

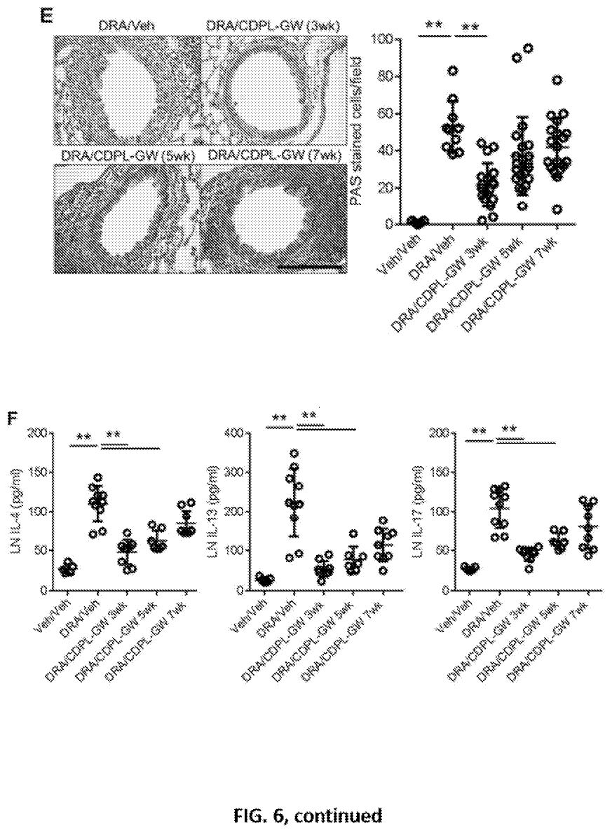

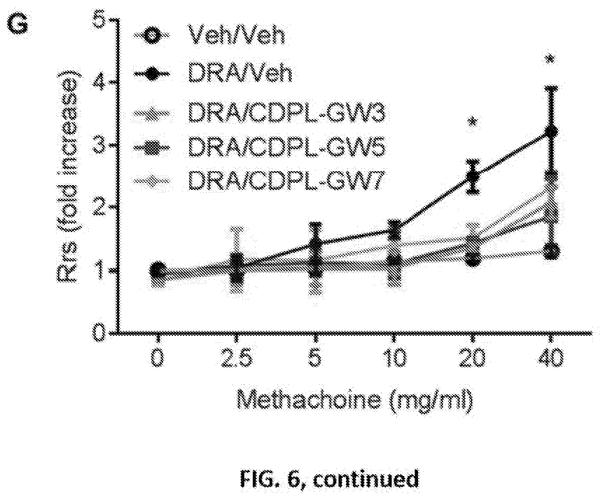

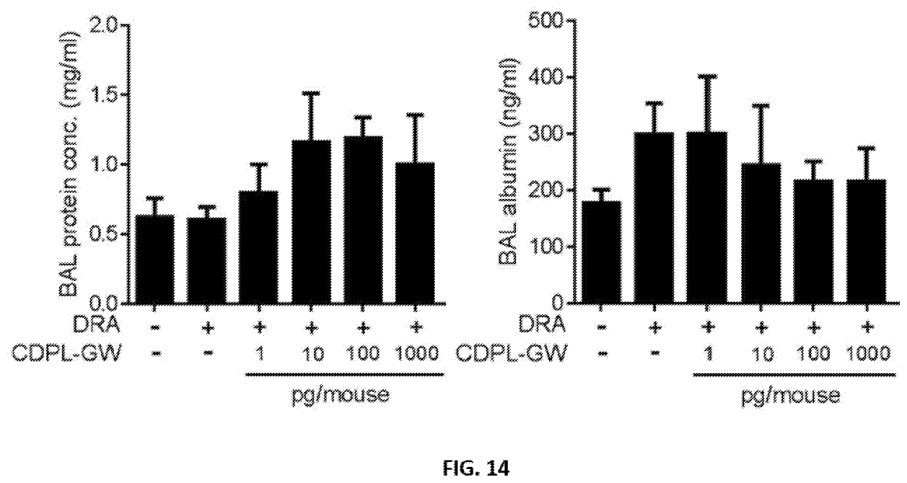

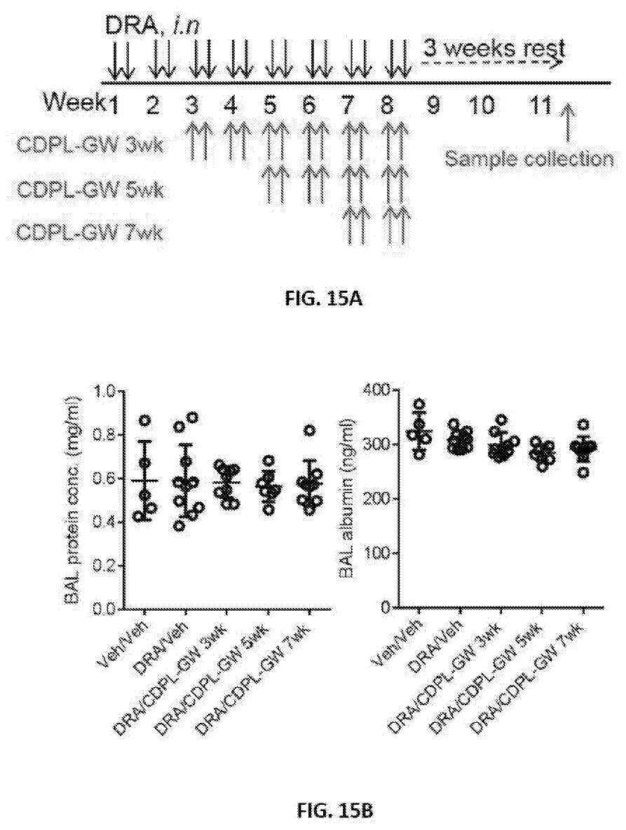

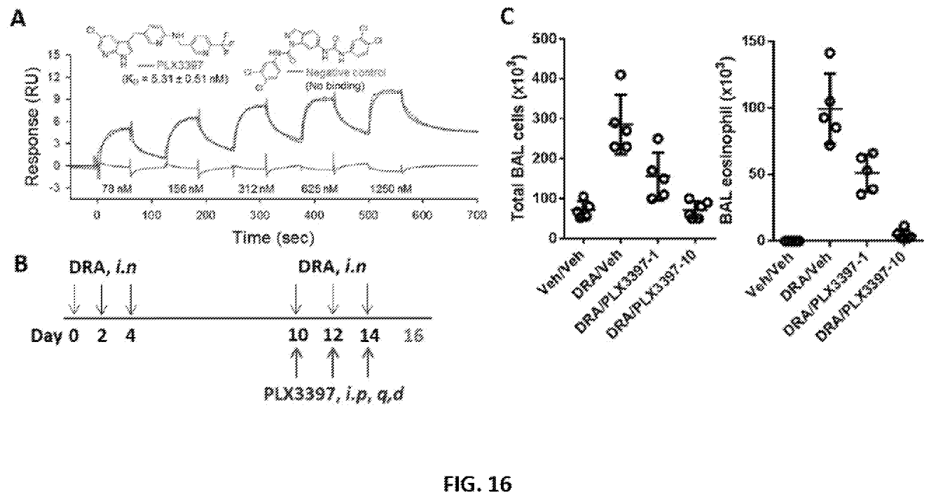

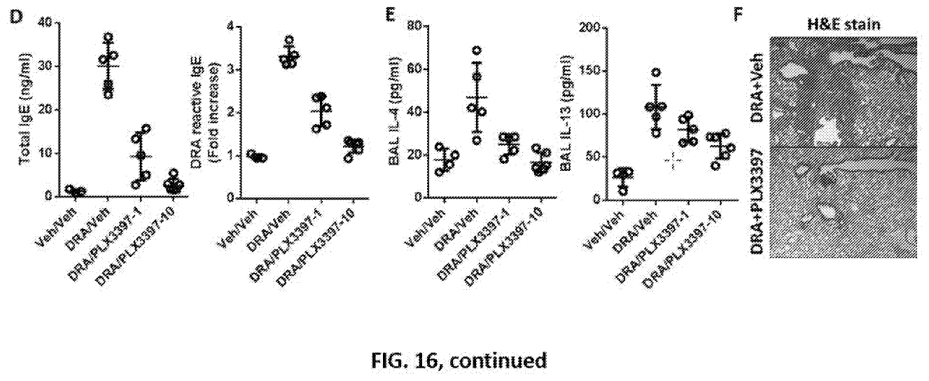

Nanoparticles carrying CSF1R inhibitor abolish chronic allergic lung inflammation in the DRA model. To measure the efficacy of intranasal CDPL-GW treatment for chronic allergic inflammation, three different experiments were performed as depicted in FIG. 14. In these experiments. CDPL-GW intranasal treatment was started at three different time points, week 3, 5 and 7, in the chronic DRA model, during the repeated DRA exposures until week 8. After 3 weeks of rest, the mice were analyzed at week 11. Intranasal CDPL-GW treatment in all three experiments effectively attenuated asthma pathologies including airway inflammatory cell recruitment, total and DRA-reactive serum IgE. BAL Th2 cytokines, lung tissue inflammation and goblet cell metaplasia, compared to the sham treated group, although the CDPL-GW 7 wk group had only modest effects (FIG. 6A-E). There was no change in the BAL concentrations of total protein and albumin (FIG. 14). CDPL-GW treatment effectively inhibited Th2 memory cell formation in the secondary LNs, although the CDPL-GW 7 wk group did not reach statistical significance (FIG. 6F). All three CDPL-GW treated groups showed reduced bronchial hyper-responsiveness, compared to the sham-treated chronic DRA group (FIG. 6G).

The disclosed study highlights the effects of blocking or minimizing allergen sensitization on development and exacerbation of asthma pathologies, respectively. Recent clinical studies indicate that sensitization to environmental allergens and excessive production of serum IgE precedes manifestation of allergic asthma in the early childhood (Hose A J, et al. J Allergy Clin Immunol 2017 139:1935-1945 e1912: Rhodes H L, et al. Am J Respir Crit Care Med 2002 165:176-180). In the cohort of a large group of newborns, the prevalence of asthma was significantly higher among ever-allergic sensitized children compared to never-sensitized ones (Ballardini N, et al. Allergy 2016 71:342-349). These data strongly suggest a causal relationship between allergen sensitization and development of clinical asthma. Therapies to reduce allergen sensitization have been tried using many different approaches. Allergen immunotherapy with allergen extracts is able to lower allergen-specific IgE as well as increase specific IgG4 levels, resulting in a robust long-lasting effect on seasonal rhinitis and peanut allergy (Durham S R, et al. J Allergy Clin Immunol 2012 129:717-725 e715; Durham S R, et al. J Allergy Clin Immunol 2010 125:131-138 e131-137; Du Toit G, et al. N Engl J Med 2015 372:803-813). Sensitization to allergen(s) occurs not only in the development of new asthma, but also in the exacerbation of established asthma. Serum IgE is increased by re-exposure to the sensitizing allergen in the mouse DRA model of allergic asthma (Moon H-G, et al. Immunity 2018 49:275-287.e275). Intervening in the continuing sensitization process has been shown to prevent asthma exacerbations in the patients already sensitized. For example, direct IgE-targeted treatment with omalizumab, a humanized monoclonal anti-IgE antibody, showed a significant benefit in controlling asthma exacerbations in inner city children who were already sensitized to cockroach (Busse W W, et al. N Engl J Med 2011364:1005-1015). Pre-seasonal treatment with omalizumab was also effective in controlling seasonal exacerbations in poorly controlled asthmatics (Teach S J, et al. J Allergy Clin Immunol 2015 136:1476-1485). Here, three animal models were examined to validate the effect of intervening in the CSF1-CSF1R pathway for preventing both the development of new allergic asthma and reversing fully established allergic lung inflammation. In all three models, data showed that blocking the CSF1-CSF1R pathway effectively abolished the sensitization process (reducing both total and allergen-reactive serum IgE) and suppressed subsequent allergic lung inflammation. These data support the concept that blocking the allergen sensitization process can effectively prevent the subsequent Th2 allergic inflammatory process regardless of the onset of allergic inflammation, and could be used as a maintenance therapy for chronic asthma.

Among DC subsets, cDC2 has been known to play an essential key role in antigen presentation in allergic lung inflammation (Plantinga M, et al. Immunity 2013 38:322-335; Moon H-G, et al. Immunity 2018 49:275-287.e275; Hammad H. et al. J Exp Med 2010 207:2097-2111). Data show a marked surge of cDC2 in BAL fluid in the DRA asthma model (Moon H-G, et al. Immunity 2018 49:275-287.e275). However, it is not fully understood how alveolar cDC2 are regulated in allergic inflammation. The CSF1-CSF1R pathway is critical for cDC2 activation through their homing to regional LNs and establishing subsequent Th2 immune reactions in response to inhaled allergens. The binding of AEC-derived CSF1 to its receptor on cDC2 facilitates the expression of chemokine receptor, CCR7 (Moon H-G, et al. Immunity 2018 49:275-287.e275). Based on this scientific premise, here it was rigorously examined whether intervening this pathway is beneficial for controlling new and established allergic lung inflammation. To examine the therapeutic effectiveness, a more clinically relevant mouse model of chronic asthma was adopted (Goplen N, et al. J Allergy Clin Immunol 2009 123:925-932.e911). Unlike the transient acute models of allergic asthma, this model shows marked inflammatory cell infiltration and prominent airway remodeling, including structural alterations, prominent tissue fibrosis and chronic inflammation, with decreased eosinophilia (FIG. 2). This model is better suited for evaluating the long term effects of asthma therapeutics on well-established asthma pathologies, in this case, treatment with anti-CSF1 antibodies and nanoparticles carrying a CSF1R antagonist.

CSF1 and its receptor (CSF1R) regulate the functions of myeloid lineage cells that play key roles in innate immune responses (Stanley E R, Chitu V. CSF-1 receptor signaling in myeloid cells. Cold Spring Harb Perspect Biol 2014 6). CSF1R is comprised of five extracellular immunoglobulin domains, a transmembrane domain, and two intracellular domains. The CSF1R gene is evolutionally well conserved and has high sequence homology between species. Especially, the intracellular domains of human and murine CSF1R genes are identical (NCBI HomologGene). In humans, the CSF1R gene is located at the chromosomal region 5q32, which is close to genes associated with genetic susceptibility to asthma including IL-4, IL-5 and ADRB2 (27-29). Furthermore, it has been reported that a polymorphism of the CSF1R gene is associated with an increased risk for asthma in humans (Shin E K. et al. Hum Genet 2010 128:293-302). Upon binding with its ligand, CSF1, the intracellular kinase domain of CSF1R undergoes auto-phosphorylation and activates multiple downstream pathways (Stanley E R, Chitu V. CSF-1 receptor signaling in myeloid cells. Cold Spring Harb Perspect Biol 2014 6). CSF1R inhibitors bind to the tyrosine residues of the intracellular kinase domain. The simulation shows that the CSF1R inhibitor, GW2580, binds two tyrosine residues (Y546 and Y665) (FIG. 9). Further studies are necessary to delineate the effect of GW2580 binding to CSF1R and modulation of downstream signaling pathways associated with allergic airway responses.

Systemic administration of CSF1R inhibitors as anti-cancer agents has been shown to have excellent clinical safety and good tolerance, even in the patients with advanced-staged malignancies (Butowski N, et al. Neuro Oncol 2016 18:557-564; Tap W D, et al. N EngI J Med 2015 373:428-437). Here, the therapeutic efficacy and potential pulmonary toxicities of locally administered CSF1R inhibitor, GW2580, was examined. The data show a decrease in BAL eosinophil count and serum IgE level with inhalational delivery of CDPL-GW at the dose of 1 ng/mouse, which is equivalent to 8,000 nanoparticles per mouse. These data indicate that the DCs involved in allergen sensitization are highly sensitive to blockade of the CSF1-CSF1R pathway. Since not only DCs but also macrophages express CSF1R, macrophage dysfunction related to CSF1R inhibition may lead to potential adverse effects. Although not being involved in antigen presentation, AMs are a first-line of defense against inhaled foreign particles. Here, the potential pulmonary toxicities of locally administered CSF1R inhibitor nanoparticles was examined. The intranasal delivery of CDPL-GW (up to 1 ng/mouse) did not increase AM cell death in terms of the number of apoptotic AMs. Various forms of pulmonary alveolar proteinosis (PAP) can develop because of impaired function of GM-CSF or its receptor (CSF2R). The studies herein examined whether CDPL-GW induced PAP in the mice, even though CSF1R is structurally different from the GM-CSF receptor and there is no known cross-reactivity between these two receptors. The data showed that there was no increase in total protein or albumin concentrations in BAL fluid, and lung pathology showed no evidence of PAP after treatment with intranasal CDPL-GW. These data suggest that the sensitivity of the CSF1R receptor to CSF1R inhibition varies depending on the cell type. A previous report showing variable expression of CSF1R among myeloid cells supports this hypothesis (Moon H-G, et al. Immunity 2018 49:275-287.e275). Of interest, blocking the CSF1-CSF1R pathway selectively inhibits Th2 memory, but Th1 and Th17 memory function remained intact, suggesting that immune defense against viral and bacterial infection may remain intact as well, although this requires further investigation. Previous reports indicate that the differentiation and survival of AMs heavily depends on GM-CSF and its receptor (Guilliams M, et al. Nat Rev Immunol 2017 17:451-460; van de Laar L, et al. Immunity 2016 44:755-768), suggesting that CSF1R could be dispensable for macrophages in the alveolar niche. Further study is needed to clarify the biological function of the CSF1 receptor on alveolar macrophages.

In summary, the disclosed data support the efficacy of a DC centered new treatment of asthma that targets the CSF1-CSF1R pathway to block the process of allergen sensitization. Because allergen sensitization is required not only for establishing new atopic asthma, but also for exacerbation of allergic inflammation in already established asthma, the approach of blocking the sensitization process could be used as a long term maintenance therapy for the treatment of asthma.

It will be apparent to those skilled in the art that various modifications and variations can be made in the present disclosure without departing from the scope or spirit of the disclosure. Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

1. A method of treating a disease or condition characterized by allergic inflammation in a subject comprising administering to the subject an effective amount of a small molecule CSF1R inhibitor.

2. The method of claim 1, wherein the CSF1R inhibitor is selected from PLX3397, DCC-3014, BLZ945, GW2580, PLX647 and ARRY-382, or a pharmaceutically acceptable salt thereof.

3. The method of claim 2, wherein CSF1R inhibitor is GW2580 or PLX3397, or a pharmaceutically acceptable salt thereof.

4. The method of claim 1, wherein an effective amount of a small molecule CSF1R inhibitor comprises a microparticle comprising the small molecule CSF1R inhibitor.

5. The method of claim 4, wherein the microparticle further comprises s-cyclodextrin conjugated epsilon-polylysine.

6. The method of claim 1, wherein the CSF1R inhibitor is administered to the patient during an acute allergic flare-up.

7. The method of claim 1, wherein the CSF1R inhibitor is administered to the patient prior to an acute allergic flare-up.

8. The method of claim 1, wherein the disease or condition characterized by allergic inflammation is selected from asthma, allergic conjunctivitis, allergic dermatitis, allergic esophagitis and allergic rhinitis.

9. The method of claim 8 wherein the CSF1R inhibitor is GW2580 or PLX3397, or a pharmaceutically acceptable salt thereof.

10. The method of claim 8, wherein the disease or condition is asthma or allergic conjunctivitis.

11. The method of claim 10, wherein the CSF1R inhibitor is administered to the patient by nasal aerosol or inhalation.

12. The method of claim 8 wherein the disease or condition is allergic conjunctivitis.

13. The method of claim 12, wherein the CSF1R inhibitor is administered to the patient by application of an ophthalmic composition comprising the CSF1R inhibitor.

14. The method of claim 8 wherein the disease or condition characterized by allergic inflammation is allergic dermatitis.

15. The method of claim 14, wherein the CSF1R inhibitor is administered to the patient by application of a topical cream or ointment comprising the CSF1R inhibitor.

16. The method of claim 8 wherein the disease or condition characterized by allergic inflammation is allergic esophagitis.

17. The method of claim 16, wherein the CSF1R inhibitor is administered to the patient by ingestion of an oral composition comprising the CSF1R inhibitor.

18. A microparticle comprising a CSF1R inhibitor or a pharmaceutically acceptable salt thereof.

19. The microparticle of claim 18, wherein the CSF1R inhibitor is selected from PLX3397, DCC-3014, BLZ945, GW2580, PLX647 and ARRY-382, or a pharmaceutically acceptable salt thereof.

20. The microparticle of claim 19, further comprising β-cyclodextrin conjugated epsilon-polylysine.
